SEQUENCE LISTING (56 sequences)

SEQ ID NO: 1 - protein, 79 aa, Homo sapiens
SEAEDASLLSFMQGYMKHATKTAKDALSSVQESQVAQQARGWVTDGFSSLKDYWSTVKDKFSEFWDLDPEVRPTSAVAA

SEQ ID NO: 2 - protein, 99 aa, Macaca fascicularis
MQPRVLLVAALLSLLASARASEAEDTSLLGFMQGYMQHATKTAKDALTSVQESQVAQQARGWVTDGFSSLKDYWSTVKDKLSGFWDLNPEAKPTLAEAA

SEQ ID NO: 3 - protein, 9 aa, Homo sapiens
FSEFWDLDP

SEQ ID NO: 4 - protein, 9 aa, Macaca fascicularis
LSGFWDLNP

SEQ ID NO: 5 - protein, 5 aa, Artificial Sequence
  source: /note=“Description of Artificial Sequence Synthetic peptide”
TYSMR

SEQ ID NO: 6 - protein, 17 aa, Artificial Sequence
  source: /note=“Description of Artificial Sequence Synthetic peptide”
SISTDGGGTAYRDSVKG

SEQ ID NO: 7 - protein, 5 aa, Artificial Sequence
  source: /note=“Description of Artificial Sequence Synthetic peptide”
AGYSD

SEQ ID NO: 8 - protein, 16 aa, Artificial Sequence
  source: /note=“Description of Artificial Sequence Synthetic peptide”
KAGQNLVHPDGKTYLY

SEQ ID NO: 9 - protein, 16 aa, Artificial Sequence
  source: /note=“Description of Artificial Sequence Synthetic peptide”
KASQNLVHSNGKTYLY

SEQ ID NO: 10 - protein, 16 aa, Artificial Sequence
  source: /note=“Description of Artificial Sequence Synthetic peptide”
KASQSLVYSDGKTYLY

SEQ ID NO: 11 - protein, 16 aa, Artificial Sequence
  source: /note=“Description of Artificial Sequence Synthetic peptide”
KATQSLVHIDGKTYLY

SEQ ID NO: 12 - protein, 16 aa, Artificial Sequence
  source: /note=“Description of Artificial Sequence Synthetic peptide”
TASQSLRHSDGRTYLY

SEQ ID NO: 13 - protein, 16 aa, Artificial Sequence
  source: /note=“Description of Artificial Sequence Synthetic peptide”
KASQSLVHPDGKTYLY

SEQ ID NO: 14 - protein, 7 aa, Artificial Sequence
  source: /note=“Description of Artificial Sequence Synthetic peptide”
QVSNRDS

SEQ ID NO: 15 - protein, 7 aa, Artificial Sequence
  source: /note=“Description of Artificial Sequence Synthetic peptide”
QVSNRGS

SEQ ID NO: 16 - protein, 7 aa, Artificial Sequence
  source: /note=“Description of Artificial Sequence Synthetic peptide”
QVSTRDS

SEQ ID NO: 17 - protein, 7 aa, Artificial Sequence
  source: /note=“Description of Artificial Sequence Synthetic peptide”
RVSTRDP

SEQ ID NO: 18 - protein, 7 aa, Artificial Sequence
  source: /note=“Description of Artificial Sequence Synthetic peptide”
QVSNRPS

SEQ ID NO: 19 - protein, 9 aa, Artificial Sequence
  source: /note=“Description of Artificial Sequence Synthetic peptide”
AQGTYWPKT

SEQ ID NO: 20 - protein, 9 aa, Artificial Sequence
  source: /note=“Description of Artificial Sequence Synthetic peptide”
AQDTYSTKT

SEQ ID NO: 21 - protein, 9 aa, Artificial Sequence
  source: /note=“Description of Artificial Sequence Synthetic peptide”
AQGTYYPLT

SEQ ID NO: 22 - protein, 16 aa, Artificial Sequence
  source: /note=“Description of Artificial Sequence Synthetic peptide”
  VARIANT (1)..(1): /replace=“Thr”
  VARIANT (3)..(3): /replace=“Ser” or “Thr”
  VARIANT (5)..(5): /replace=“Ser”
  VARIANT (7)..(7): /replace=“Arg”
  VARIANT (8)..(8): /replace=“Tyr”
  VARIANT (9)..(9): /replace=“Pro” or “Ser”
  VARIANT (10)..(10): /replace=“Asn”
  VARIANT (12)..(12): /replace=“Arg”
  SITE (1)..(16): /note=“Variant residues given in the sequence have no preference with respect to those in the annotations for variant positions”
KAGQNLVHIDGKTYLY

SEQ ID NO: 23 - protein, 7 aa, Artificial Sequence
  source: /note=“Description of Artificial Sequence Synthetic peptide”
  VARIANT (1)..(1): /replace=“Gly”
  VARIANT (4)..(4): /replace=“Thr”
  VARIANT (6)..(6): /replace=“Gly” or “Pro”
  SITE (1)..(7): /note=“Variant residues given in the sequence have no preference with respect to those in the annotations for variant positions”
DVSNRDS

SEQ ID NO: 24 - protein, 9 aa, Artificial Sequence
  source: /note=“Description of Artificial Sequence Synthetic peptide”
  VARIANT (3)..(3): /replace=“Gly”
  VARIANT (6)..(6): /replace=“Trp” or “Tyr”
  VARIANT (7)..(7): /replace=“Thr”
  VARIANT (8)..(8): /replace=“Leu”
  SITE (1)..(9): /note=“Variant residues given in the sequence have no preference with respect to those in the annotations for variant positions”
AQDTYSPKT

SEQ ID NO: 25 - protein, 114 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSS

SEQ ID NO: 26 - protein, 112 aa, Artificial Sequence
  source: /note=“Description of Artificial Sequence Synthetic polypeptide”
ATMLTQSPGSLSVVPGESASISCKTSQGLVHSDGKTYFYWFLQKPGQSPQQLIYQVSNRASGVPDRFTGSGSGTDFTLKISGVKAEDAGVYYCAQGTYYPHTFGSGTRLEIK

SEQ ID NO: 27 - protein, 112 aa, Artificial Sequence
  source: /note=“Description of Artificial Sequence Synthetic polypeptide”
DVVLTQTPGSLSVVPGESASISCKAGQNLVHPDGKTYLYWLLQKPGQSPQRLIYQVSNRDSGVPDRFTGSGSGTDFTLKISGVKVEDAGVYYCAQGTYWPKTFGQGTKLEIK

SEQ ID NO: 28 - protein, 112 aa, Artificial Sequence
  source: /note=“Description of Artificial Sequence Synthetic polypeptide”
DVVLTQTPGSLSVVPGESASISCKASQNLVHSNGKTYLYWLLQKPGQSPQRLIYQVSNRGSEVPDRFTGSGSGTDFTLKISGVKAEDAGVYYCAQGTYWPKTFGQGTKLEIK

SEQ ID NO: 29 - protein, 112 aa, Artificial Sequence
  source: /note=“Description of Artificial Sequence Synthetic polypeptide”
DVVLTQTPGSLSVVPGESASISCKASQSLVYSDGKTYLYWLLQKPGQSPQRLIYQVSNRDSGVPDRFTGSGSGTDFTLKISGVKVEDAGVYYCAQGTYWPKTFGQGTKLEIK

SEQ ID NO: 30 - protein, 112 aa, Artificial Sequence
  source: /note=“Description of Artificial Sequence Synthetic polypeptide”
DVVLTQTPGSLSVVPGESASISCKATQSLVHIDGKTYLYWLLQKPGQSPQRLIYQVSTRDSGVPDRFTGAGSGAEFTLKISGVKAEDAGVYYCAQDTYSTKTFGQGTKLEIK

SEQ ID NO: 31 - protein, 112 aa, Artificial Sequence
  source: /note=“Description of Artificial Sequence Synthetic polypeptide”
DVVLTQTPGSLSVVPGESASISCTASQSLRHSDGRTYLYWLRQKPGQSPQRLIKRVSTRDPGVPDRFTGSGSGTDFTLKISGVRAEDAGVYYCAQGTYYPLTFGQGTKVELK

SEQ ID NO: 32 - protein, 112 aa, Artificial Sequence
  source: /note=“Description of Artificial Sequence Synthetic polypeptide”
DVVLTQTPGSLSVVPGEPASVSCKASQSLVHPDGKTYLYWLLQKPGQSPQRLIYQVSNRGSGVPDRFTGSGSGTDFTLEISGVKAEDAGVYYCAQGTYWPKTFGQGTKLEIK

SEQ ID NO: 33 - protein, 112 aa, Artificial Sequence
  source: /note=“Description of Artificial Sequence Synthetic polypeptide”
DVVLTQTPGSLSVVPGGSASISCKASQSLVYSDGKTYLYWLRQKPGQSPQRLIYQVSNRPSGVPDRFTGSGSGTDFTLKISGVKAEDAGVYYCAQDTYSTKTFGQGTKLEIK

SEQ ID NO: 34 - protein, 444 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 443 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 444 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSIHTDGGGTAYRDSVKGRFTISRDNAKNTLYLQMNNLKPEDTAIYYCVIAGYSDWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 443 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 444 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 39 - protein, 443 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 40 - protein, 444 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 41 - protein, 443 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 42 - protein, 441 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 440 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 44 - protein, 441 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 440 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 46 - protein, 441 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALKFHYTQKSLSLSLGK

SEQ ID NO: 47 - protein, 440 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALKFHYTQKSLSLSLG

SEQ ID NO: 48 - protein, 441 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 49 - protein, 440 aa, Artificial Sequence
  source: /note=“Description of Artificial Sequence Synthetic polypeptide”
QLQLVESGGGLVQPGGSLRLSCAASGFTFGTYSMRWVRQVPRKALEWVSSISTDGGGTAYRDSVKGRFTISRDNAKNTLYLQMNNLKPEDTAIYYCVIAGYSDWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 50 - protein, 219 aa, Artificial Sequence
  source: /note=“Description of Artificial Sequence Synthetic polypeptide”
DVVLTQTPGSLSVVPGESASISCKASQNLVHSNGKTYLYWLLQKPGQSPQRLIYQVSNRGSEVPDRFTGSGSGTDFTLKISGVKAEDAGVYYCAQGTYWPKTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 219 aa, Artificial Sequence
  source: /note=“Description of Artificial Sequence Synthetic polypeptide”
DVVLTQTPGSLSVVPGESASISCKAGQNLVHPDGKTYLYWLLQKPGQSPQRLIYQVSNRDSGVPDRFTGSGSGTDFTLKISGVKVEDAGVYYCAQGTYWPKTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 219 aa, Artificial Sequence
  source: /note=“Description of Artificial Sequence Synthetic polypeptide”
DVVLTQTPGSLSVVPGESASISCKASQSLVYSDGKTYLYWLLQKPGQSPQRLIYQVSNRDSGVPDRFTGSGSGTDFTLKISGVKVEDAGVYYCAQGTYWPKTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 219 aa, Artificial Sequence
  source: /note=“Description of Artificial Sequence Synthetic polypeptide”
DVVLTQTPGSLSVVPGESASISCKATQSLVHIDGKTYLYWLLQKPGQSPQRLIYQVSTRDSGVPDRFTGAGSGAEFTLKISGVKAEDAGVYYCAQDTYSTKTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 219 aa, Artificial Sequence
  source: /note=“Description of Artificial Sequence Synthetic polypeptide”
DVVLTQTPGSLSVVPGESASISCTASQSLRHSDGRTYLYWLRQKPGQSPQRLIKRVSTRDPGVPDRFTGSGSGTDFTLKISGVRAEDAGVYYCAQGTYYPLTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 219 aa, Artificial Sequence
  source: /note=“Description of Artificial Sequence Synthetic polypeptide”
DVVLTQTPGSLSVVPGEPASVSCKASQSLVHPDGKTYLYWLLQKPGQSPQRLIYQVSNRGSGVPDRFTGSGSGTDFTLEISGVKAEDAGVYYCAQGTYWPKTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 219 aa, Artificial Sequence
  source: /note=“Description of Artificial Sequence Synthetic polypeptide”
DVVLTQTPGSLSVVPGGSASISCKASQSLVYSDGKTYLYWLRQKPGQSPQRLIYQVSNRPSGVPDRFTGSGSGTDFTLKISGVKAEDAGVYYCAQDTYSTKTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0347865 A1
(43) Pub. Date: Nov. 11, 2021

(54) ANTIBODIES SPECIFIC FOR HUMAN AND CYNOMOLGUS APOC3 AND METHODS OF USE THEREOF

(71) Applicant: Staten Biotechnology B.V., Nijmegen (NL)

(72) Inventors: Paul DASILVA-JARDINE, Guilford, CT (US); Hans DE HAARD, Oudelande (NL)

(21) Appl. No.: 17/221,271

(22) Filed: Apr. 2, 2021

Related U.S. Application Data
(63) Continuation of application No. PCT/IB2019/058403, filed on Oct. 3, 2019.
(60) Provisional application No. 62/740,798, filed on Oct. 3, 2018.

(51) Int. Cl. C07K 16/18; A61P 3/06; A61K 39/395; A61K 45/06; A61K 31/397

(52) U.S. Cl. CPC C07K 16/18; A61P 3/06; A61K 2039/505; A61K 45/06; A61K 31/397; A61K 39/3955

(57) ABSTRACT

The instant disclosure provides antibodies that specifically bind to ApoC3 (e.g., human or cynomolgus ApoC3) and antagonizes ApoC3 function. Also provided are pharmaceutical compositions comprising these antibodies, nucleic acids encoding these antibodies, expression vectors and host cells for making these antibodies, and methods of treating a subject using these antibodies.

### RELATED APPLICATIONS

This application claims the benefit of U.S. Provisional Application No. 62/740,798, filed Oct. 3, 2018, which is incorporated by reference herein in its entirety.

### FIELD

The instant disclosure relates to antibodies that specifically bind to human and cynomolgus monkey ApoC3 and methods of using the same.

### BACKGROUND

Elevated blood triglyceride levels (hypertriglyceridemia) are a causal factor for atherosclerosis, and increase the risk of cardiovascular events, such as cardiovascular death, angina, myocardial infarction, and stroke.

ApoC3 is a protein that circulates at very high concentrations (greater than 10 μM) in the blood, mostly bound to triglyceride rich lipoprotein (TRL), TRL remnants, and high density lipoprotein. ApoC3 appears to be an important regulator of blood triglyceride levels. For example, ApoC3 levels in humans have been shown to positively correlate with blood triglyceride levels, with elevated ApoC3 levels being associated with hypertriglyceridemia. In addition, ApoC3 has been shown to inhibit the activity of lipoprotein lipase (an enzyme that hydrolyses triglycerides in TRL) and also to inhibit hepatic uptake of TRL remnants, both of which cause elevation of blood triglyceride levels.

Several therapies have been approved for the treatment hypertriglyceridemia, such as fibrates, niacin, and omega-3 fatty acids. However these therapies are only modestly effective at lowering plasma triglycerides. Accordingly, there is a need in the art for improved therapies for lowering plasma triglycerides.

### SUMMARY

The instant disclosure provides antibodies that specifically bind to ApoC3 (e.g., human or cynomolgus ApoC3) and inhibit ApoC3 function. Also provided are pharmaceutical compositions comprising these antibodies, nucleic acids encoding these antibodies, expression vectors and host cells for making these antibodies, and methods of treating a subject using these antibodies. The antibodies disclosed herein are capable of binding to human and cynomolgus monkey ApoC3, and are particularly advantageous in that they can attenuate the ability of ApoC3 to inhibit TRL uptake by hepatocytes, and can cause a rapid and sustained decrease in the serum levels of ApoC3 when administered to a human or cynomolgus monkey subject. Accordingly, the disclosed anti-ApoC3 antibodies are useful for the treatment and prevention of hypertriglyceridemia and associated diseases (e.g., cardiovascular disease and pancreatitis).

Accordingly, in one aspect, the instant disclosure provides an isolated antibody that specifically binds to human and cynomologus monkey ApoC3, wherein the antibody specifically binds to an epitope within the amino acid sequence FSEFWDLDP (SEQ ID NO: 3). In certain embodiments, the antibody specifically binds to an epitope within the amino acid sequence LSGFWDLNP (SEQ ID NO: 4). In certain embodiments, the antibody specifically binds to at least one of the amino acids at position 2, 5, or 6 of SEQ ID NO: 3. In certain embodiments, the antibody specifically binds to the amino acids at: (a) positions 2 and 5 of SEQ ID NO: 3; (b) positions 2 and 6 of SEQ ID NO: 3; (c) positions 5 and 6 of SEQ ID NO: 3; or (d) positions 2, 5, and 6 of SEQ ID NO: 3.

In another aspect, the instant disclosure provides an isolated antibody that specifically binds to human and cynomologus monkey ApoC3, comprising a heavy chain variable region comprising complementarity determining regions CDRH1, CDRH2 and CDRH3, and a light chain variable region comprising complementarity determining regions CDRL1, CDRL2 and CDRL3, wherein:

(a) CDRH1 comprises the amino acid sequence TYSMR (SEQ ID NO: 5);

(b) CDRH2 comprises the amino acid sequence SISTDGGGTAYRDSVKG (SEQ ID NO: 6);

(c) CDRH3 comprises the amino acid sequence AGYSD (SEQ ID NO: 7);

(d) CDRL1 comprises the amino acid sequence X1AX2QX3LX4X5X6X7GX8TYLY (SEQ ID NO: 22), wherein

X1 is K or T,

X2 is G, S or T,

X3 is N or S,

X4 is V or R,

X5 is H or Y,

X6 is I, P or S,

X7 is D or N, and

X8 is K or R;

(e) CDRL2 comprises the amino acid sequence X1VSX2RX3S (SEQ ID NO: 23), wherein

X1 is D or G;

X2 is N or T; and

X3 is D, G or P; and

(f) CDRL3 comprises the amino acid sequence AQX1TYX2X3X4T (SEQ ID NO: 24), wherein

X1 is D or G;

X2 is S, W or Y;

X3 is P or T;

X4 is K or L.

In certain embodiments: (a) CDRL1 comprises an amino acid sequence selected from the group consisting of SEQ ID NO: 8, 9, 10, 11, 12 and 13; (b) CDRL2 comprises an amino acid sequence selected from the group consisting of SEQ ID NO: 14, 15, 16, 17, and 18; and/or (c) CDRL3 comprises an amino acid sequence selected from the group consisting of SEQ ID NO: 19, 20, and 21.

In another aspect, the instant disclosure provides an isolated antibody that specifically binds to human and cynomologus monkey ApoC3, comprising a heavy chain variable region comprising complementarity determining regions CDRH1, CDRH2 and CDRH3, and a light chain variable region comprising complementarity determining regions CDRL1, CDRL2 and CDRL3, wherein CDRH1, CDRH2, CDRH3, CDRL1, CDRL2, and CDRL3 comprise the amino acid sequences set forth in SEQ ID NOs: 5, 6, 7, 8, 14, and 19; 5, 6, 7, 9, 15, and 19; 5, 6, 7, 10, 14, and 19; 5, 6, 7, 11, 16, and 20; 5, 6, 7, 12, 17, and 21; 5, 6, 7, 13, 15, and 19; or 5, 6, 7, 10, 18, and 20, respectively. In certain embodiments, the antibody comprises a light chain variable region comprising an amino acid sequence selected from the group consisting of SEQ ID NOs: 27-33.

In another aspect, the instant disclosure provides an isolated antibody that specifically binds to ApoC3, the antibody comprising a light chain variable region comprising an amino acid sequence selected from the group consisting of SEQ ID NOs: 27-33.

In another aspect, the instant disclosure provides an isolated antibody that specifically binds to ApoC3, the antibody comprising a heavy chain variable region and a light chain variable region, wherein the heavy chain variable region and the light chain variable region, respectively, comprise the amino acid sequences set forth in SEQ ID NOs: 25 and 27, 25 and 28, 25 and 29, 25 and 30, 25 and 31, 25 and 32, or 25 and 33.

In certain embodiments, the antibody comprises a human lambda or human kappa light chain constant region. In certain embodiments, the antibody comprises a light chain comprising the amino acid sequence set forth in SEQ ID NO: 50, 51, 52, 53, 54, 55, or 56.

In certain embodiments, the antibody comprises a heavy chain constant region, optionally a human IgG1, IgG2, or IgG4 constant region. In certain embodiments, the constant region is a variant of a wild type human immunoglobulin heavy chain constant region, and wherein the variant human immunoglobulin heavy chain constant region has an increased affinity for human neonatal Fc receptor (FcRn) at pH 6 relative to the affinity of the corresponding wild type human immunoglobulin heavy chain constant region for human FcRn at pH 6.

In certain embodiments, the heavy chain constant region comprises the amino acids K, F, and Y at EU positions 433, 434, and 436, respectively. In certain embodiments, the heavy chain constant region comprises the amino acids Y, T, and E at EU positions 252, 254, and 256, respectively. In certain embodiments, the heavy chain constant region comprises the amino acids L and S at EU positions 428 and 434, respectively. In certain embodiments, the heavy chain constant region is an IgG4 constant region comprising the amino acid P at EU position 228. In certain embodiments, the antibody comprises a heavy chain comprising of the amino acid sequence set forth in SEQ ID NO: 34, 35, 36, 37, 38, 39, 40, 41, 42, 43, 44, 45, 46, 47, 48, or 49.

In another aspect, the instant disclosure provides an isolated antibody that specifically binds to ApoC3, the antibody comprising a heavy chain and a light chain, wherein the amino acid sequences of the heavy chain and the light chain, respectively, comprise or consist of the amino acid sequences set forth in SEQ ID NOs: 34 and 50, 35 and 50, 36 and 50, 37 and 50, 38 and 50, 39 and 50, 40 and 50, 41 and 50, 42 and 50, 43 and 50, 44 and 50, 45 and 50, 46 and 50, 47 and 50, 48 and 50, or 49 and 50.

In certain embodiments, the antibody is capable of binding to lipid-bound ApoC3. In certain embodiments, the antibody attenuates the ability of ApoC3 to inhibit hepatocyte uptake of very low density lipoprotein (VLDL). In certain embodiments, the antibody is capable of increasing the rate of clearance of ApoC3 from the blood in a subject. In certain embodiments, the antibody is capable of reducing the level of ApoC3 in the blood in a subject. In certain embodiments, the antibody is capable of inhibiting post-prandial lipemia in a subject.

In another aspect, the instant disclosure provides a pharmaceutical composition comprising an antibody as disclosed herein and a pharmaceutically acceptable carrier.

In another aspect, the instant disclosure provides a polynucleotide encoding the heavy chain variable region and/or the light chain variable region of an antibody disclosed herein. In another aspect, the instant disclosure provides an expression vector comprising a polynucleotide disclosed herein. In another aspect, the instant disclosure provides a host cell comprising an expression vector disclosed herein.

In another aspect, the instant disclosure provides a method for producing an antibody that binds to ApoC3, the method comprising culturing a host cell disclosed herein under conditions that allow expression of the antibody.

In another aspect, the instant disclosure provides a method for inhibiting the activity of ApoC3 in the blood of a subject, the method comprising administering to the subject an effective amount of an antibody or pharmaceutical composition disclosed herein.

In another aspect, the instant disclosure provides a method for reducing triglyceride levels in the blood of a subject, the method comprising administering to the subject an effective amount of an antibody or pharmaceutical composition disclosed herein. In another aspect, the instant disclosure provides a method for inhibiting post-prandial lipemia in a subject, the method comprising administering to the subject an effective amount of an antibody or pharmaceutical composition disclosed herein. In another aspect, the instant disclosure provides a method for treating hypertriglyceridemia in a subject, the method comprising administering to the subject an effective amount of an antibody or pharmaceutical composition disclosed herein. In another aspect, the instant disclosure provides a method for treating chylomicronemia in a subject, the method comprising administering to the subject an effective amount of an antibody or pharmaceutical composition disclosed herein.

In another aspect, the instant disclosure provides a method for reducing the risk of cardiovascular disease in a subject with hypertriglyceridemia, the method comprising administering to the subject an effective amount of an antibody or pharmaceutical composition disclosed herein. In certain embodiments, the cardiovascular disease is myocardial infarction. In certain embodiments, the cardiovascular disease is angina. In certain embodiments, the cardiovascular disease is stroke. In certain embodiments, the cardiovascular disease is atherosclerosis.

In certain embodiments of the foregoing aspects relating to treatment methods, the antibody reduces the levels of chylomicron or chylomicron remnants in the blood of the subject. In certain embodiments, the subject is receiving an additional lipid lowering agent. In certain embodiments, the additional lipid lowering agent is an HMG-CoA reductase inhibitor. In certain embodiments, the HMG-CoA reductase inhibitor is atorvastatin, fluvastatin, lovastatin, pitavastatin, pravastatin, rosuvastatin or simvastatin. In certain embodiments, the additional lipid lowering agent is a PCSK9 inhibitor. In certain embodiments, the PCSK9 inhibitor is alirocumab, evolocumab, or bococizumab. In certain embodiments, the additional lipid lowering agent is ezetimibe. In certain embodiments, the additional lipid lowering agent is a combination of ezetimibe and an HMG-CoA reductase inhibitor. In certain embodiments, the additional lipid lowering agent is a combination of ezetimibe, an HMG-CoA reductase inhibitor, and a PCSK9 inhibitor.

### BRIEF DESCRIPTION OF THE DRAWINGS

FIGS. 1A and 1B are graphs showing the levels of human ApoC3 in an AAV8-huApoC3 mouse model treated with Hyhel5 control antibody, pre-germline, pH-dependent 5E5VH5_VL8 antibody, and 29A06 test antibody. FIG. 1A shows the percent change in human ApoC3 levels following antibody administration. FIG. 1B shows human ApoC3 levels (μM) in mouse plasma.

FIG. 2 is a graph showing the percent change in mouse ApoC3 levels in an AAV8-huApoC3 mouse model treated with Hyhel5 control antibody, pre-germline, pH-dependent 5E5VH5_VL8 antibody, and 29A06 test antibody.

FIGS. 3A and 3B are graphs showing the plasma triglyceride levels in an AAV8-huApoC3 mouse model treated with Hyhel5 control antibody, pre-germline, pH-dependent 5E5VH5_VL8 antibody, and 29A06 test antibody. FIG. 3A shows the percent change in triglyceride levels following antibody administration. FIG. 3B shows triglyceride levels (mg/dL) in mouse plasma.

FIG. 4 is a graph showing the levels of IgG (μg/mL) in an AAV8-huApoC3 mouse model treated with Hyhel5 control antibody, pre-germline, pH-dependent 5E5VH5_VL8 antibody, and 29A06 test antibody.

FIGS. 5A and 5B are graphs showing the levels of ApoC3 in cynomolgus monkeys treated with three doses of 29A06-NHance antibody. FIG. 5A shows the percent change in cynomolgus ApoC3 levels following antibody administration. FIG. 5B shows cynomolgus ApoC3 levels (μM) in plasma.

FIG. 6 is graph showing cynomolgus ApoB levels (mg/dL) in animals treated with three doses of 29A06-NHance antibody.

FIGS. 7A and 7B are graphs showing the levels of serum triglycerides in cynomolgus monkeys treated with three doses of 29A06-NHance antibody. FIG. 7A shows the percent change in triglyceride levels following antibody administration. FIG. 7B shows triglyceride levels (mg/dL) in cynomolgus serum.

FIG. 8 is a graph showing the levels of IgG (μg/mL) in cynomolgus monkeys treated with three doses of 29A06-NHance antibody.

### DETAILED DESCRIPTION

The instant disclosure provides antibodies that specifically bind to human and cynomolgus monkey ApoC3 and inhibit ApoC3 function. Also provided are pharmaceutical compositions comprising these antibodies, nucleic acids encoding these antibodies, expression vectors and host cells for making these antibodies, and methods of treating a subject using these antibodies. The antibodies disclosed herein are capable of binding to, human and cynomolgus monkey ApoC3, and are particularly advantageous in that they can attenuate the ability of ApoC3 to inhibit TRL uptake by hepatocytes, and can cause a rapid and sustained decrease in the serum levels of ApoC3 when administered to a human or cynomolgus monkey subject. Accordingly, the disclosed anti-ApoC3 antibodies are useful for the treatment and prevention of hypertriglyceridemia and associated diseases (e.g., cardiovascular disease and pancreatitis).

### 1. Definitions

As used herein, the term “ApoC3” refers to Apolipoprotein C3 protein. In certain embodiments, the ApoC3 is human ApoC3. An exemplary human ApoC3 amino acid sequence is set forth in RefSeq accession number NP_000031.1. The mature amino acid sequence of NP_000031.1 is as follows:

(SEQ ID NO: 1)
SEAEDASLLSFMQGYMKHATKTAKDALSSVQESQVAQQARGWVTDGFSS
LKDYWSTVKDKFSEFWDLDPEVRPTSAVAA.

In certain embodiments, the ApoC3 is cynomolgus monkey ApoC3. An exemplary cynomolgus monkey ApoC3 amino acid sequence is set forth in RefSeq accession number XP_005579787.1. The mature amino acid sequence of XP_005579787.1 is as follows:

(SEQ ID NO: 2)
MQPRVLLVAALLSLLASARASEAEDTSLLGFMQGYMQHATKTAKDALTS
VQESQVAQQARGWVTDGFSSLKDYWSTVKDKLSGFWDLNPEAKPTLAEA
A.

As used herein, the terms “antibody” and “antibodies” include full length antibodies, antigen-binding fragments of full length antibodies, and molecules comprising antibody CDRs, VH regions or VL regions. Examples of antibodies include monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), scFv-Fcs, camelid antibodies (e.g., llama antibodies), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies disclosed herein refer to polyclonal antibody populations. Antibodies can be of any type (e.g., IgG, IgE, IgM, IgD, IgA or IgY), any class (e.g., IgG₁, IgG₂, IgG₃, IgG₄, IgA₁ or IgA₂), or any subclass (e.g., IgG₂ₐ or IgG_{2b}) of immunoglobulin molecule. In certain embodiments, antibodies disclosed herein are IgG antibodies, or a class (e.g., human IgG₁ or IgG₄) or subclass thereof In a specific embodiment, the antibody is a humanized monoclonal antibody.

As used herein, the term “isolated antibody” refers to an antibody that has been identified and separated and/or recovered from at least one component of its natural environment. The term “isolated antibody” includes an antibody in situ within a recombinant host cell.

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), by Chothia et al., J. Mol. Biol. 196:901-917 (1987), and by MacCallum et al., J. Mol. Biol. 262:732-745 (1996), all of which are incorporated by reference in their entireties, where the definitions include overlapping or subsets of amino acid residues when compared against each other. In certain embodiments, the term “CDR” is a CDR as defined by Kabat et al., J. Biol. Chem. 252, 6609-6616 (1977) and Kabat et al., Sequences of protein of immunological interest. (1991). CDRH1, CDRH2 and CDRH3 denote the heavy chain CDRs, and CDRL1, CDRL2 and CDRL3 denote the light chain CDRs.

As used herein, the term “framework (FR) amino acid residues” refers to those amino acids in the framework region of an immunoglobulin chain. The term “framework region” or “FR region” as used herein, includes the amino acid residues that are part of the variable region, but are not part of the CDRs (e.g., using the Kabat definition of CDRs).

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

As used herein, the terms “constant region” and “constant domain” are interchangeable and are common in the art. The constant region is an antibody portion, e.g., a carboxyl terminal portion of a light or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

As used herein, the term “EU position” refers to the amino acid position according to the EU numbering convention for the constant regions of an antibody, as described in Edelman, G. M. et al., Proc. Natl. Acad. USA, 63, 78-85 (1969) and Kabat et al., in “Sequences of Proteins of Immunological Interest”, U.S. Dept. Health and Human Services, 5th edition, 1991, each of which is herein incorporated by reference in its entirety.

As used herein, the term “specifically binds to” refers to the ability of an antibody to bind to an antigen with an dissociation constant (K_{D}) of less than about 1×10^{˜6} M, 1×10⁻⁷ M, 1×10⁻⁸ M, 1×10⁻⁹ M, 1×10⁻¹⁰ M, 1×10⁻¹¹ M, 1×10⁻¹² M, or less, or bind to an antigen with an affinity that is at least two-fold greater than its affinity for a nonspecific antigen.

As used herein, an “epitope” refers to a localized region of an antigen to which an antibody can specifically bind. An epitope can be, for example, contiguous amino acids of a polypeptide (a linear or contiguous epitope) or an epitope can, for example, be formed from two or more non-contiguous regions of a polypeptide or polypeptides (a conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), peptide scanning assays, or mutagenesis mapping (e.g., site-directed mutagenesis mapping).

As used herein, the term “treat,” “treating,” and “treatment” refer to therapeutic or preventative measures disclosed herein. The methods of “treatment” employ administration of an anti-ApoC3 antibody to a subject having a disease or disorder, or predisposed to having such a disease or disorder, in order to prevent, cure, delay, reduce the severity of, reduce the risk of developing, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal (e.g., cynomolgus monkey).

As used herein, the term “or” means and/or.

As used herein, the terms “about” and “approximately,” when used to modify a numeric value or numeric range, indicate that deviations of 5% to 10% above and 5% to 10% below the value or range remain within the intended meaning of the recited value or range.

### 2. Anti-ApoC3 Antibodies

In one aspect, the instant disclosure provides an isolated antibody that specifically binds to human and cynomologus monkey (Macaca fascicularis) ApoC3, wherein the antibody specifically binds to an epitope within the human ApoC3 amino acid sequence FSEFWDLDP (SEQ ID NO: 3). In certain embodiments, the antibody specifically binds to at least one of the amino acids at position 2, 5, or 6 of SEQ ID NO: 3. For example, in certain embodiments, the antibody specifically binds to positions 2 and 5 of SEQ ID NO: 3. In certain embodiments, the antibody specifically binds to positions 2 and 6 of SEQ ID NO: 3. In certain embodiments, the antibody specifically binds to positions 5 and 6 of SEQ ID NO: 3. In certain embodiments, the antibody specifically binds to positions 2, 5, and 6 of SEQ ID NO: 3. In certain embodiments, the antibody specifically binds to at least one of the amino acids at position 2, 5, or 6 of SEQ ID NO: 4. In certain embodiments, the antibody also specifically binds to an epitope within the cynomolgus monkey amino acid sequence LSGFWDLNP (SEQ ID NO: 4). For example, in certain embodiments, the antibody specifically binds to positions 2 and 5 of SEQ ID NO: 4. In certain embodiments, the antibody specifically binds to positions 2 and 6 of SEQ ID NO: 4. In certain embodiments, the antibody specifically binds to positions 5 and 6 of SEQ ID NO: 4. In certain embodiments, the antibody specifically binds to positions 2, 5, and 6 of SEQ ID NO: 4.

The amino acid sequences of exemplary anti-ApoC3 antibodies are set forth in Tables 1-5, herein.

TABLE 1
Heavy chain CDR amino acid sequences of
exemplary anti-ApoC3 antibodies.

| | | SEQ | | SEQ | | SEQ |
|---|---|---|---|---|---|---|
| | | ID | | ID | | ID |
| Clone | CDRH1 | NO | CDRH2 | NO | CDRH3 | NO |
| 5E5 | TYSMR | 5 | SISTDGGGTAYRDSVKG | 6 | AGYSD | 7 |

TABLE 2
Light chain CDR amino acid sequences of
exemplary anti-ApoC3 antibodies.

| | | SEQ | | SEQ | | SEQ |
|---|---|---|---|---|---|---|
| | | ID | | ID | | ID |
| Clone | CDRL1 | NO | CDRL2 | NO | CDRL3 | NO |
| 29B03 | KAGQNLVHPDGKTYLY | 8 | QVSNRDS | 14 | AQGTYWPKT | 19 |
| 29A06 | KASQNLVHSNGKTYLY | 9 | QVSNRGS | 15 | AQGTYWPKT | 19 |
| 29A05 | KASQSLVYSDGKTYLY | 10 | QVSNRDS | 14 | AQGTYWPKT | 19 |
| 29G02 | KATQSLVHIDGKTYLY | 11 | QVSTRDS | 16 | AQDTYSTKT | 20 |
| 30D10 | TASQSLRHSDGRTYLY | 12 | RVSTRDP | 17 | AQGTYYPLT | 21 |
| 29F10 | KASQSLVHPDGKTYLY | 13 | QVSNRGS | 15 | AQGTYWPKT | 19 |
| 29G11 | KASQSLVYSDGKTYLY | 10 | QVSNRPS | 18 | AQDTYSTKT | 20 |

TABLE 3
Light chain CDR consensus amino acid sequences

| | | SEQ |
|---|---|---|
| | | ID |
| CDR | Amino acid Sequence | NO |
| CDRL1 | X₁AX₂QX₃LX₄X₅X₆X₇GX₈TYLY,wherein: | 22 |
| consensus | X₁ is K or T; | |
| | X₂ is G, S or T; | |
| | X₃ is N or S; | |
| | X₄ is V or R; | |
| | X₅ is H or Y; | |
| | X₆ is I, P or S; | |
| | X₇ is D or N; and | |
| | X₈ is K or R. | |
| CDRL2 | X₁VSX₂RX₃S, wherein: | 23 |
| consensus | X₁ is D or G; | |
| | X₂ is N or T; and | |
| | X₃ is D, G or P. | |
| CDRL3 | AQX₁TYX₂X₃X₄T, wherein: | 24 |
| consensus | X₁ is D or G; | |
| | X₂ is S, W or Y; | |
| | X₃ is P or T; | |
| | X₄ is K or L. | |

TABLE 4
VH and VL amino acid sequences of exemplary
anti-ApoC3 antibodies.

| | | SEQ |
|---|---|---|
| | | ID |
| VH/VL | Amino acid Sequence | NO |
| 5E5 VH | QLQLVESGGGLVQPGGSLRLSCAASGFTFG | 25 |
| | TYSMRWVRQVPRKALEWVSSISTDGGGTAY | |
| | RDSVKGRFTISRDNAKNTLYLQMNNLKPED | |
| | TAIYYCVIAGYSDWGQGTQVTVSS | |
| 5E5 VL | ATMLTQSPGSLSVVPGESASISCKTSQGLV | 26 |
| | HSDGKTYFYWFLQKPGQSPQQLIYQVSNRA | |
| | SGVPDRFTGSGSGTDFTLKISGVKAEDAGV | |
| | YYCAQGTYYPHTFGSGTRLEIK | |
| 29B03 VL | DVVLTQTPGSLSVVPGESASISCKAGQNLV | 27 |
| | HPDGKTYLYWLLQKPGQSPQRLIYQVSNRD | |
| | SGVPDRFTGSGSGTDFTLKISGVKVEDAGV | |
| | YYCAQGTYWPKTFGQGTKLEIK | |
| 29A06 VL | DVVLTQTPGSLSVVPGESASISCKASQNLV | 28 |
| | HSNGKTYLYWLLQKPGQSPQRLIYQVSNRG | |
| | SEVPDRFTGSGSGTDFTLKISGVKAEDAGV | |
| | YYCAQGTYWPKTFGQGTKLEIK | |
| 29A05 VL | DVVLTQTPGSLSVVPGESASISCKASQSLV | 29 |
| | YSDGKTYLYWLLQKPGQSPQRLIYQVSNRD | |
| | SGVPDRFTGSGSGTDFTLKISGVKVEDAGV | |
| | YYCAQGTYWPKTFGQGTKLEIK | |
| 29G02 VL | DVVLTQTPGSLSVVPGESASISCKATQSLV | 30 |
| | HIDGKTYLYWLLQKPGQSPQRLIYQVSTRD | |
| | SGVPDRFTGAGSGAEFTLKISGVKAEDAGV | |
| | YYCAQDTYSTKTFGQGTKLEIK | |
| 30D10 VL | DVVLTQTPGSLSVVPGESASISCTASQSLR | 31 |
| | HSDGRTYLYWLRQKPGQSPQRLIKRVSTRD | |
| | PGVPDRFTGSGSGTDFTLKISGVRAEDAGV | |
| | YYCAQGTYYPLTFGQGTKVELK | |
| 29F10 VL | DVVLTQTPGSLSVVPGEPASVSCKASQSLV | 32 |
| | HPDGKTYLYWLLQKPGQSPQRLIYQVSNRG | |
| | SGVPDRFTGSGSGTDFTLEISGVKAEDAGV | |
| | YYCAQGTYWPKTFGQGTKLEIK | |
| 29G11 VL | DVVLTQTPGSLSVVPGGSASISCKASQSLV | 33 |
| | YSDGKTYLYWLRQKPGQSPQRLIYQVSNRP | |
| | SGVPDRFTGSGSGTDFTLKISGVKAEDAGV | |
| | YYCAQDTYSTKTFGQGTKLEIK | |

TABLE 5
Full heavy chain and light chain sequences of exemplary
anti-ApoC3 antibodies.

| Antibody | | SEQ ID |
|---|---|---|
| chain | Amino Acid Sequence | NO |
| 29A06 | QLQLVESGGGLVQPGGSLRLSCAASGFTFGTYSMRWVRQVPR | 34 |
| IgG1 | KALEWVSSISTDGGGTAYRDSVKGRFTISRDNAKNTLYLQMNN | |
| heavy | LKPEDTAIYYCVIAGYSDWGQGTQVTVSSASTKGPSVFPLAPSS | |
| chain | KSTSGGTAALGCLVKDYFPEPVTVSWNSGALTSGVHTFPAVLQ | |
| | SSGLYSLSSVVTVPSSSLGTQTYICNVNHKPSNTKVDKKVEPKS | |
| | CDKTHTCPPCPAPELLGGPSVFLFPPKPKDTLMISRTPEVTCVVV | |
| | DVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYKT | |
| | TPPVLDSDGSFFLYSKLTVDKSRWQQGNVFSCSVMHEALHNH | |
| | YTQKSLSLSPGK | |
| 29A06 | QLQLVESGGGLVQPGGSLRLSCAASGFTFGTYSMRWVRQVPR | 35 |
| IgG1 | KALEWVSSISTDGGGTAYRDSVKGRFTISRDNAKNTLYLQMNN | |
| heavy | LKPEDTAIYYCVIAGYSDWGQGTQVTVSSASTKGPSVFPLAPSS | |
| chain | KSTSGGTAALGCLVKDYFPEPVTVSWNSGALTSGVHTFPAVLQ | |
| (minus C- | SSGLYSLSSVVTVPSSSLGTQTYICNVNHKPSNTKVDKKVEPKS | |
| term K) | CDKTHTCPPCPAPELLGGPSVFLFPPKPKDTLMISRTPEVTCVVV | |
| | DVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYKT | |
| | TPPVLDSDGSFFLYSKLTVDKSRWQQGNVFSCSVMHEALHNH | |
| | YTQKSLSLSPG | |
| 29A06 | QLQLVESGGGLVQPGGSLRLSCAASGFTFGTYSMRWVRQVPR | 36 |
| IgG1 | KALEWVSSIHTDGGGTAYRDSVKGRFTISRDNAKNTLYLQMN | |
| heavy | NLKPEDTAIYYCVIAGYSDWGQGTQVTVSSASTKGPSVFPLAPS | |
| chain | SKSTSGGTAALGCLVKDYFPEPVTVSWNSGALTSGVHTFPAVL | |
| YTE | QSSGLYSLSSVVTVPSSSLGTQTYICNVNHKPSNTKVDKKVEPK | |
| | SCDKTHTCPPCPAPELLGGPSVFLFPPKPKDTLYITREPEVTCVV | |
| | VDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| 29A06 | QLQLVESGGGLVQPGGSLRLSCAASGFTFGTYSMRWVRQVPR | 37 |
| IgG1 | KALEWVSSISTDGGGTAYRDSVKGRFTISRDNAKNTLYLQMNN | |
| heavy | LKPEDTAIYYCVIAGYSDWGQGTQVTVSSASTKGPSVFPLAPSS | |
| chain | KSTSGGTAALGCLVKDYFPEPVTVSWNSGALTSGVHTFPAVLQ | |
| YTE | SSGLYSLSSVVTVPSSSLGTQTYICNVNHKPSNTKVDKKVEPKS | |
| (minus C- | CDKTHTCPPCPAPELLGGPSVFLFPPKPKDTLYITREPEVTCVVV | |
| term K) | DVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYKT | |
| | TPPVLDSDGSFFLYSKLTVDKSRWQQGNVFSCSVMHEALHNH | |
| | YTQKSLSLSPG | |
| 29A06 | QLQLVESGGGLVQPGGSLRLSCAASGFTFGTYSMRWVRQVPR | 38 |
| IgG1 | KALEWVSSISTDGGGTAYRDSVKGRFTISRDNAKNTLYLQMNN | |
| heavy | LKPEDTAIYYCVIAGYSDWGQGTQVTVSSASTKGPSVFPLAPSS | |
| chain | KSTSGGTAALGCLVKDYFPEPVTVSWNSGALTSGVHTFPAVLQ | |
| Nhance | SSGLYSLSSVVTVPSSSLGTQTYICNVNHKPSNTKVDKKVEPKS | |
| | CDKTHTCPPCPAPELLGGPSVFLFPPKPKDTLMISRTPEVTCVVV | |
| | DVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYKT | |
| | TPPVLDSDGSFFLYSKLTVDKSRWQQGNVFSCSVMHEALKFHY | |
| | TQKSLSLSPGK | |
| 29A06 | QLQLVESGGGLVQPGGSLRLSCAASGFTFGTYSMRWVRQVPR | 39 |
| IgG1 | KALEWVSSISTDGGGTAYRDSVKGRFTISRDNAKNTLYLQMNN | |
| heavy | LKPEDTAIYYCVIAGYSDWGQGTQVTVSSASTKGPSVFPLAPSS | |
| chain | KSTSGGTAALGCLVKDYFPEPVTVSWNSGALTSGVHTFPAVLQ | |
| Nhance | SSGLYSLSSVVTVPSSSLGTQTYICNVNHKPSNTKVDKKVEPKS | |
| (minus C- | CDKTHTCPPCPAPELLGGPSVFLFPPKPKDTLMISRTPEVTCVVV | |
| term K) | DVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYKT | |
| | TPPVLDSDGSFFLYSKLTVDKSRWQQGNVFSCSVMHEALKFHY | |
| | TQKSLSLSPG | |
| 29A06 | QLQLVESGGGLVQPGGSLRLSCAASGFTFGTYSMRWVRQVPR | 40 |
| IgG1 | KALEWVSSISTDGGGTAYRDSVKGRFTISRDNAKNTLYLQMNN | |
| heavy | LKPEDTAIYYCVIAGYSDWGQGTQVTVSSASTKGPSVFPLAPSS | |
| chain | KSTSGGTAALGCLVKDYFPEPVTVSWNSGALTSGVHTFPAVLQ | |
| Xtend | SSGLYSLSSVVTVPSSSLGTQTYICNVNHKPSNTKVDKKVEPKS | |
| | CDKTHTCPPCPAPELLGGPSVFLFPPKPKDTLMISRTPEVTCVVV | |
| | DVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYKT | |
| | TPPVLDSDGSFFLYSKLTVDKSRWQQGNVFSCSVLHEALHSHY | |
| | TQKSLSLSPGK | |
| 29A06 | QLQLVESGGGLVQPGGSLRLSCAASGFTFGTYSMRWVRQVPR | 41 |
| IgG1 | KALEWVSSISTDGGGTAYRDSVKGRFTISRDNAKNTLYLQMNN | |
| heavy | LKPEDTAIYYCVIAGYSDWGQGTQVTVSSASTKGPSVFPLAPSS | |
| chain | KSTSGGTAALGCLVKDYFPEPVTVSWNSGALTSGVHTFPAVLQ | |
| Xtend | SSGLYSLSSVVTVPSSSLGTQTYICNVNHKPSNTKVDKKVEPKS | |
| (minus C- | CDKTHTCPPCPAPELLGGPSVFLFPPKPKDTLMISRTPEVTCVVV | |
| term K) | DVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYKT | |
| | TPPVLDSDGSFFLYSKLTVDKSRWQQGNVFSCSVLHEALHSHY | |
| | TQKSLSLSPG | |
| 29A06 | QLQLVESGGGLVQPGGSLRLSCAASGFTFGTYSMRWVRQVPR | 42 |
| IgG4 | KALEWVSSISTDGGGTAYRDSVKGRFTISRDNAKNTLYLQMNN | |
| S228P | LKPEDTAIYYCVIAGYSDWGQGTQVTVSSASTKGPSVFPLAPCS | |
| heavy | RSTSESTAALGCLVKDYFPEPVTVSWNSGALTSGVHTFPAVLQS | |
| chain | SGLYSLSSVVTVPSSSLGTKTYTCNVDHKPSNTKVDKRVESKY | |
| | GPPCPPCPAPEFLGGPSVFLFPPKPKDTLMISRTPEVTCVVVDVS | |
| | QEDPEVQFNWYVDGVEVHNAKTKPREEQFNSTYRVVSVLTVL | |
| | HQDWLNGKEYKCKVSNKGLPSSIEKTISKAKGQPREPQVYTLPP | |
| | SQEEMTKNQVSLTCLVKGFYPSDIAVEWESNGQPENNYKTTPP | |
| | VLDSDGSFFLYSRLTVDKSRWQEGNVFSCSVMHEALHNHYTQ | |
| | KSLSLSLGK | |
| 29A06 | QLQLVESGGGLVQPGGSLRLSCAASGFTFGTYSMRWVRQVPR | 43 |
| IgG4 | KALEWVSSISTDGGGTAYRDSVKGRFTISRDNAKNTLYLQMNN | |
| S228P | LKPEDTAIYYCVIAGYSDWGQGTQVTVSSASTKGPSVFPLAPCS | |
| heavy | RSTSESTAALGCLVKDYFPEPVTVSWNSGALTSGVHTFPAVLQS | |
| chain | SGLYSLSSVVTVPSSSLGTKTYTCNVDHKPSNTKVDKRVESKY | |
| (minus C- | GPPCPPCPAPEFLGGPSVFLFPPKPKDTLMISRTPEVTCVVVDVS | |
| term K) | QEDPEVQFNWYVDGVEVHNAKTKPREEQFNSTYRVVSVLTVL | |
| | HQDWLNGKEYKCKVSNKGLPSSIEKTISKAKGQPREPQVYTLPP | |
| | SQEEMTKNQVSLTCLVKGFYPSDIAVEWESNGQPENNYKTTPP | |
| | VLDSDGSFFLYSRLTVDKSRWQEGNVFSCSVMHEALHNHYTQ | |
| | KSLSLSLG | |
| 29A06 | QLQLVESGGGLVQPGGSLRLSCAASGFTFGTYSMRWVRQVPR | 44 |
| IgG4 | KALEWVSSISTDGGGTAYRDSVKGRFTISRDNAKNTLYLQMNN | |
| S228P | LKPEDTAIYYCVIAGYSDWGQGTQVTVSSASTKGPSVFPLAPCS | |
| heavy | RSTSESTAALGCLVKDYFPEPVTVSWNSGALTSGVHTFPAVLQS | |
| chain | SGLYSLSSVVTVPSSSLGTKTYTCNVDHKPSNTKVDKRVESKY | |
| YTE | GPPCPPCPAPEFLGGPSVFLFPPKPKDTLYITREPEVTCVVVDVS | |
| | QEDPEVQFNWYVDGVEVHNAKTKPREEQFNSTYRVVSVLTVL | |
| | HQDWLNGKEYKCKVSNKGLPSSIEKTISKAKGQPREPQVYTLPP | |
| | SQEEMTKNQVSLTCLVKGFYPSDIAVEWESNGQPENNYKTTPP | |
| | VLDSDGSFFLYSRLTVDKSRWQEGNVFSCSVMHEALHNHYTQ | |
| | KSLSLSLGK | |
| 29A06 | QLQLVESGGGLVQPGGSLRLSCAASGFTFGTYSMRWVRQVPR | 45 |
| IgG4 | KALEWVSSISTDGGGTAYRDSVKGRFTISRDNAKNTLYLQMNN | |
| S228P | LKPEDTAIYYCVIAGYSDWGQGTQVTVSSASTKGPSVFPLAPCS | |
| heavy | RSTSESTAALGCLVKDYFPEPVTVSWNSGALTSGVHTFPAVLQS | |
| chain YTE | SGLYSLSSVVTVPSSSLGTKTYTCNVDHKPSNTKVDKRVESKY | |
| (minus C- | GPPCPPCPAPEFLGGPSVFLFPPKPKDTLYITREPEVTCVVVDVS | |
| term K) | QEDPEVQFNWYVDGVEVHNAKTKPREEQFNSTYRVVSVLTVL | |
| | HQDWLNGKEYKCKVSNKGLPSSIEKTISKAKGQPREPQVYTLPP | |
| | SQEEMTKNQVSLTCLVKGFYPSDIAVEWESNGQPENNYKTTPP | |
| | VLDSDGSFFLYSRLTVDKSRWQEGNVFSCSVMHEALHNHYTQ | |
| | KSLSLSLG | |
| 29A06 | QLQLVESGGGLVQPGGSLRLSCAASGFTFGTYSMRWVRQVPR | 46 |
| IgG4 | KALEWVSSISTDGGGTAYRDSVKGRFTISRDNAKNTLYLQMNN | |
| S228P | LKPEDTAIYYCVIAGYSDWGQGTQVTVSSASTKGPSVFPLAPCS | |
| heavy | RSTSESTAALGCLVKDYFPEPVTVSWNSGALTSGVHTFPAVLQS | |
| chain | SGLYSLSSVVTVPSSSLGTKTYTCNVDHKPSNTKVDKRVESKY | |
| Nhance | GPPCPPCPAPEFLGGPSVFLFPPKPKDTLMISRTPEVTCVVVDVS | |
| | QEDPEVQFNWYVDGVEVHNAKTKPREEQFNSTYRVVSVLTVL | |
| | HQDWLNGKEYKCKVSNKGLPSSIEKTISKAKGQPREPQVYTLPP | |
| | SQEEMTKNQVSLTCLVKGFYPSDIAVEWESNGQPENNYKTTPP | |
| | VLDSDGSFFLYSRLTVDKSRWQEGNVFSCSVMHEALKFHYTQ | |
| | KSLSLSLGK | |
| 29A06 | QLQLVESGGGLVQPGGSLRLSCAASGFTFGTYSMRWVRQVPR | 47 |
| IgG4 | KALEWVSSISTDGGGTAYRDSVKGRFTISRDNAKNTLYLQMNN | |
| S228P | LKPEDTAIYYCVIAGYSDWGQGTQVTVSSASTKGPSVFPLAPCS | |
| heavy | RSTSESTAALGCLVKDYFPEPVTVSWNSGALTSGVHTFPAVLQS | |
| chain | SGLYSLSSVVTVPSSSLGTKTYTCNVDHKPSNTKVDKRVESKY | |
| Nhance | GPPCPPCPAPEFLGGPSVFLFPPKPKDTLMISRTPEVTCVVVDVS | |
| (minus C- | QEDPEVQFNWYVDGVEVHNAKTKPREEQFNSTYRVVSVLTVL | |
| term K) | HQDWLNGKEYKCKVSNKGLPSSIEKTISKAKGQPREPQVYTLPP | |
| | SQEEMTKNQVSLTCLVKGFYPSDIAVEWESNGQPENNYKTTPP | |
| | VLDSDGSFFLYSRLTVDKSRWQEGNVFSCSVMHEALKFHYTQ | |
| | KSLSLSLG | |
| 29A06 | QLQLVESGGGLVQPGGSLRLSCAASGFTFGTYSMRWVRQVPR | 48 |
| IgG4 | KALEWVSSISTDGGGTAYRDSVKGRFTISRDNAKNTLYLQMNN | |
| S228P | LKPEDTAIYYCVIAGYSDWGQGTQVTVSSASTKGPSVFPLAPCS | |
| heavy | RSTSESTAALGCLVKDYFPEPVTVSWNSGALTSGVHTFPAVLQS | |
| chain | SGLYSLSSVVTVPSSSLGTKTYTCNVDHKPSNTKVDKRVESKY | |
| Xtend | GPPCPPCPAPEFLGGPSVFLFPPKPKDTLMISRTPEVTCVVVDVS | |
| | QEDPEVQFNWYVDGVEVHNAKTKPREEQFNSTYRVVSVLTVL | |
| | HQDWLNGKEYKCKVSNKGLPSSIEKTISKAKGQPREPQVYTLPP | |
| | SQEEMTKNQVSLTCLVKGFYPSDIAVEWESNGQPENNYKTTPP | |
| | VLDSDGSFFLYSRLTVDKSRWQEGNVFSCSVLHEALHSHYTQK | |
| | SLSLSLGK | |
| 29A06 | QLQLVESGGGLVQPGGSLRLSCAASGFTFGTYSMRWVRQVPR | 49 |
| IgG4 | KALEWVSSISTDGGGTAYRDSVKGRFTISRDNAKNTLYLQMNN | |
| S228P | LKPEDTAIYYCVIAGYSDWGQGTQVTVSSASTKGPSVFPLAPCS | |
| heavy | RSTSESTAALGCLVKDYFPEPVTVSWNSGALTSGVHTFPAVLQS | |
| chain | SGLYSLSSVVTVPSSSLGTKTYTCNVDHKPSNTKVDKRVESKY | |
| Xtend | GPPCPPCPAPEFLGGPSVFLFPPKPKDTLMISRTPEVTCVVVDVS | |
| (minus C- | QEDPEVQFNWYVDGVEVHNAKTKPREEQFNSTYRVVSVLTVL | |
| term K) | HQDWLNGKEYKCKVSNKGLPSSIEKTISKAKGQPREPQVYTLPP | |
| | SQEEMTKNQVSLTCLVKGFYPSDIAVEWESNGQPENNYKTTPP | |
| | VLDSDGSFFLYSRLTVDKSRWQEGNVFSCSVLHEALHSHYTQK | |
| | SLSLSLG | |
| 29A06 | DVVLTQTPGSLSVVPGESASISCKASQNLVHSNGKTYLYVVLLQ | 50 |
| Kappa | KPGQSPQRLIYQVSNRGSEVPDRFTGSGSGTDFTLKISGVKAED | |
| light | AGVYYCAQGTYWPKTFGQGTKLEIKRTVAAPSVFIFPPSDEQLK | |
| chain | SGTASVVCLLNNFYPREAKVQWKVDNALQSGNSQESVTEQDS | |
| | KDSTYSLSSTLTLSKADYEKHKVYACEVTHQGLSSPVTKSFNR | |
| | GEC | |
| 29B03 | DVVLTQTPGSLSVVPGESASISCKAGQNLVHPDGKTYLYWLLQ | 51 |
| Kappa | KPGQSPQRLIYQVSNRDSGVPDRFTGSGSGTDFTLKISGVKVED | |
| light | AGVYYCAQGTYWPKTFGQGTKLEIKRTVAAPSVFIFPPSDEQLK | |
| chain | SGTASVVCLLNNFYPREAKVQWKVDNALQSGNSQESVTEQDS | |
| | KDSTYSLSSTLTLSKADYEKHKVYACEVTHQGLSSPVTKSFNR | |
| | GEC | |
| 29A05 | DVVLTQTPGSLSVVPGESASISCKASQSLVYSDGKTYLYWLLQ | 52 |
| Kappa | KPGQSPQRLIYQVSNRDSGVPDRFTGSGSGTDFTLKISGVKVED | |
| light | AGVYYCAQGTYWPKTFGQGTKLEIKRTVAAPSVFIFPPSDEQLK | |
| chain | SGTASVVCLLNNFYPREAKVQWKVDNALQSGNSQESVTEQDS | |
| | KDSTYSLSSTLTLSKADYEKHKVYACEVTHQGLSSPVTKSFNR | |
| | GEC | |
| 29G02 | DVVLTQTPGSLSVVPGESASISCKATQSLVHIDGKTYLYWLLQK | 53 |
| Kappa | PGQSPQRLIYQVSTRDSGVPDRFTGAGSGAEFTLKISGVKAEDA | |
| light | GVYYCAQDTYSTKTFGQGTKLEIKRTVAAPSVFIFPPSDEQLKS | |
| chain | GTASVVCLLNNFYPREAKVQWKVDNALQSGNSQESVTEQDSK | |
| | DSTYSLSSTLTLSKADYEKHKVYACEVTHQGLSSPVTKSFNRGE | |
| | C | |
| 30D10 | DVVLTQTPGSLSVVPGESASISCTASQSLRHSDGRTYLYWLRQK | 54 |
| Kappa | PGQSPQRLIKRVSTRDPGVPDRFTGSGSGTDFTLKISGVRAEDA | |
| light | GVYYCAQGTYYPLTFGQGTKVELKRTVAAPSVFIFPPSDEQLKS | |
| chain | GTASVVCLLNNFYPREAKVQWKVDNALQSGNSQESVTEQDSK | |
| | DSTYSLSSTLTLSKADYEKHKVYACEVTHQGLSSPVTKSFNRGE | |
| | C | |
| 29F10 | DVVLTQTPGSLSVVPGEPASVSCKASQSLVHPDGKTYLYWLLQ | 55 |
| Kappa | KPGQSPQRLIYQVSNRGSGVPDRFTGSGSGTDFTLEISGVKAED | |
| light | AGVYYCAQGTYWPKTFGQGTKLEIKRTVAAPSVFIFPPSDEQLK | |
| chain | SGTASVVCLLNNFYPREAKVQWKVDNALQSGNSQESVTEQDS | |
| | KDSTYSLSSTLTLSKADYEKHKVYACEVTHQGLSSPVTKSFNR | |
| | GEC | |
| 29G11 | DVVLTQTPGSLSVVPGGSASISCKASQSLVYSDGKTYLYWLRQ | 56 |
| Kappa | KPGQSPQRLIYQVSNRPSGVPDRFTGSGSGTDFTLKISGVKAED | |
| light | AGVYYCAQDTYSTKTFGQGTKLEIKRTVAAPSVFIFPPSDEQLK | |
| chain | SGTASVVCLLNNFYPREAKVQWKVDNALQSGNSQESVTEQDS | |
| | KDSTYSLSSTLTLSKADYEKHKVYACEVTHQGLSSPVTKSFNR | |
| | GEC | |

In certain embodiments, the instant disclosure provides an isolated antibody that specifically binds to ApoC3 (e.g., human or cynomolgus ApoC3), the antibody comprising a VH domain comprising one, two, or all three of the CDRs of a VH domain set forth in Table 4 herein. In certain embodiments, the antibody comprises the CDRH1 of the VH domain set forth in Table 4. In certain embodiments, the antibody comprises the CDRH2 of the VH domain set forth in Table 4. In certain embodiments, the antibody comprises the CDRH3 of the VH domain set forth in Table 4.

In certain embodiments, the instant disclosure provides an isolated antibody that specifically binds to ApoC3 (e.g., human or cynomolgus ApoC3), the antibody comprising a VL domain comprising one, two, or all three of the CDRs of a VL domain disclosed in Table 4 herein. In certain embodiments, the antibody comprises the CDRL1 of one of VL domains set forth in Table 4. In certain embodiments, the antibody comprises the CDRL2 of one of the VL domains set forth in Table 4. In certain embodiments, the antibody comprises the CDRL3 of one of the VL domains set forth in Table 4.

In certain embodiments, the instant disclosure provides an isolated antibody that specifically binds to ApoC3 (e.g., human or cynomolgus ApoC3), the antibody comprising a heavy chain variable region having complementarity determining regions CDRH1, CDRH2 and CDRH3, and a light chain variable region having complementarity determining regions CDRL1, CDRL2 and CDRL3, wherein CDRH1, CDRH2, CDRH3, CDRL1, CDRL2, and CDRL3 comprise the amino acid sequences of the CDRH1, CDRH2, CDRH3, CDRL1, CDRL2, and CDRL3 regions, respectively, of an antibody set forth in Tables 1-4.

In certain embodiments, the CDRs of an antibody can be determined according to Kabat et al., J. Biol. Chem. 252, 6609-6616 (1977) and Kabat et al., Sequences of protein of immunological interest (1991). In certain embodiments, the light chain CDRs of an antibody are determined according to Kabat and the heavy chain CDRs of an antibody are determined according to MacCallum (supra).

In certain embodiments, the CDRs of an antibody can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDRH1 loop is present at heavy chain amino acids 26 to 32, 33, or 34, the Chothia CDRH2 loop is present at heavy chain amino acids 52 to 56, and the Chothia CDRH3 loop is present at heavy chain amino acids 95 to 102, while the Chothia CDRL1 loop is present at light chain amino acids 24 to 34, the Chothia CDRL2 loop is present at light chain amino acids 50 to 56, and the Chothia CDRL3 loop is present at light chain amino acids 89 to 97. The end of the Chothia CDRH1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain embodiments, the CDRs of an antibody can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, CDRH1 is at positions 26 to 35, CDRH2 is at positions 51 to 57, CDRH3 is at positions 93 to 102, CDRL1 is at positions 27 to 32, CDRL2 is at positions 50 to 52, and CDRL3 is at positions 89 to 97.

In certain embodiments, the CDRs of an antibody can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.).

In certain embodiments, the CDRs of an antibody can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001).

In certain embodiments, the instant disclosure provides an isolated antibody that specifically binds to ApoC3 (e.g., human or cynomolgus ApoC3), wherein the antibody comprises a heavy chain variable region comprising the CDRH1, CDRH2, and CDRH3 region amino acid sequences of a VH domain set forth in Table 4, and a light chain variable region comprising the CDRL1, CDRL2, and CDRL3 region amino acid sequences of a VL domain set forth in Table 2, wherein each CDR is independently defined in accordance with the Kabat, Chothia, IMGT, MacCallum, or AbM definition of a CDR, as disclosed herein.

In certain embodiments, the instant disclosure provides an isolated antibody that specifically binds to ApoC3 (e.g., human or cynomolgus ApoC3), the antibody comprising a heavy chain variable region having complementarity determining regions CDRH1, CDRH2 and CDRH3, and a light chain variable region having complementarity determining regions CDRL1, CDRL2 and CDRL3, wherein:

(a) CDRH1 comprises the amino acid sequence of TYSMR (SEQ ID NO: 5);
(b) CDRH2 comprises the amino acid sequence of SISTDGGGTAYRDSVKG (SEQ ID NO: 6);
(c) CDRH3 comprises the amino acid sequence of AGYSD (SEQ ID NO: 7);
(d) CDRL1 comprises the amino acid sequence of X₁AX₂QX₃LX₄X₅X₆X₇GX₈TYLY, wherein
X₁ is K or T; X₂ is G, S or T; X₃ is N or S; X₄ is V or R; X₅ is H or Y; X₆ is P or S; X₇ is D or N; and X₈ is K or R (SEQ ID NO: 22);
(e) CDRL2 comprises the amino acid sequence of X₁VSX₂RX₃S, wherein X₁ is D or G; X₂ is N or T; and X₃ is D, G or P (SEQ ID NO: 23); and
(f) CDRL3 comprises the amino acid sequence of AQX₁TYX₂X₃X₄T, wherein X₁ is D or G; X₂ is 5, W or Y; X₃ is P or T; X₄ is K or L (SEQ ID NO: 24).

In certain embodiments, CDRL1 comprises the amino acid sequence of

(SEQ ID NO: 8)
KAGQNLVHPDGKTYLY,
(SEQ ID NO: 9)
KASQNLVHSNGKTYLY,
(SEQ ID NO: 10)
KASQSLVYSDGKTYLY,
(SEQ ID NO: 11)
KATQSLVHIDGKTYLY,
(SEQ ID NO: 12)
TASQSLRHSDGRTYLY,
or
(SEQ ID NO: 13)
KASQSLVHPDGKTYLY.

In certain embodiments, CDRL2 comprises the amino acid sequence of QVSNRDS (SEQ ID NO: 14), QVSNRGS (SEQ ID NO: 15), QVSTRDS (SEQ ID NO: 16), RVSTRDP (SEQ ID NO: 17), or QVSNRPS (SEQ ID NO: 18).

In certain embodiments, CDRL3 comprises the amino acid sequence of

(SEQ ID NO: 19)
AQGTYWPKT,
(SEQ ID NO: 20)
AQDTYSTKT,
or
(SEQ ID NO: 21)
AQGTYYPLT.

In certain embodiments, the instant disclosure provides an isolated antibody that specifically binds to ApoC3 (e.g., human or cynomolgus ApoC3), wherein the antibody comprises a VH domain comprising the CDRH1, CDRH2 and CDRH3 amino acid sequences set forth in SEQ ID NOs: 5, 6, and 7, respectively.

In certain embodiments, the instant disclosure provides an isolated antibody that specifically binds to ApoC3 (e.g., human or cynomolgus ApoC3), wherein the antibody comprises a VL domain comprising the CDRL1, CDRL2 and CDRL3 amino acid sequences set forth in SEQ ID NOs: 8, 14, and 19; 9, 15, and 19; 10, 14, and 19; 11, 16, and 20; 12, 17, and 21; 13, 15, and 19; or 10, 18, and 20, respectively. In certain embodiments, the VL domain comprises the CDRL1, CDRL2 and CDRL3 amino acid sequences set forth in SEQ ID NOs: 8, 14, and 19, respectively. In certain embodiments, the VL domain comprises the CDRL1, CDRL2 and CDRL3 amino acid sequences set forth in SEQ ID NOs: 9, 15, and 19, respectively. In certain embodiments, the VL domain comprises the CDRL1, CDRL2 and CDRL3 amino acid sequences set forth in SEQ ID NOs: 10, 14, and 19, respectively. In certain embodiments, the VL domain comprises the CDRL1, CDRL2 and CDRL3 amino acid sequences set forth in SEQ ID NOs: 11, 16, and 20, respectively. In certain embodiments, the VL domain comprises the CDRL1, CDRL2 and CDRL3 amino acid sequences set forth in SEQ ID NOs: 12, 17, and 21, respectively. In certain embodiments, the VL domain comprises the CDRL1, CDRL2 and CDRL3 amino acid sequences set forth in SEQ ID NOs: 13, 15, and 19, respectively. In certain embodiments, the VL domain comprises the CDRL1, CDRL2 and CDRL3 amino acid sequences set forth in SEQ ID NOs: 10, 18, and 20, respectively.

In certain embodiments, the instant disclosure provides an isolated antibody that specifically binds to ApoC3 (e.g., human or cynomolgus ApoC3), wherein the antibody comprises a heavy chain variable region comprising CDRH1, CDRH2, and CDRH3 regions, and a light chain variable region comprising CDRL1, CDRL2, and CDRL3 regions, wherein the CDRH1, CDRH2, CDRH3, CDRL1, CDRL2, and CDRL3 regions comprise the amino acid sequences set forth in SEQ ID NOs: 5, 6, 7, 8, 14, and 19; 5, 6, 7, 9, 15, and 19; 5, 6, 7, 10, 14, and 19; 5, 6, 7, 11, 16, and 20; 5, 6, 7, 12, 17, and 21; 5, 6, 7, 13, 15, and 19; or 5, 6, 7, 10, 18, and 20, respectively. In certain embodiments, the CDRH1, CDRH2, CDRH3, CDRL1, CDRL2, and CDRL3 regions comprise the amino acid sequences set forth in SEQ ID NOs: 5, 6, 7, 8, 14, and 19, respectively. In certain embodiments, the CDRH1, CDRH2, CDRH3, CDRL1, CDRL2, and CDRL3 regions comprise the amino acid sequences set forth in SEQ ID NOs: 5, 6, 7, 9, 15, and 19, respectively. In certain embodiments, the CDRH1, CDRH2, CDRH3, CDRL1, CDRL2, and CDRL3 regions comprise the amino acid sequences set forth in SEQ ID NOs: 5, 6, 7, 10, 14, and 19, respectively. In certain embodiments, the CDRH1, CDRH2, CDRH3, CDRL1, CDRL2, and CDRL3 regions comprise the amino acid sequences set forth in SEQ ID NOs: 5, 6, 7, 11, 16, and 20, respectively. In certain embodiments, the CDRH1, CDRH2, CDRH3, CDRL1, CDRL2, and CDRL3 regions comprise the amino acid sequences set forth in SEQ ID NOs: 5, 6, 7, 12, 17, and 21, respectively. In certain embodiments, the CDRH1, CDRH2, CDRH3, CDRL1, CDRL2, and CDRL3 regions comprise the amino acid sequences set forth in SEQ ID NOs: 5, 6, 7, 13, 15, and 19, respectively. In certain embodiments, the CDRH1, CDRH2, CDRH3, CDRL1, CDRL2, and CDRL3 regions comprise the amino acid sequences set forth in SEQ ID NOs: 5, 6, 7, 10, 18, and 20, respectively.

In certain embodiments, the instant disclosure provides an isolated antibody that specifically binds to ApoC3 (e.g., human or cynomolgus ApoC3), comprising a heavy chain variable region comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to the amino acid sequence set forth in SEQ ID NO: 25. In certain embodiments, the antibody comprises a heavy chain variable region comprising the amino acid sequence set forth in SEQ ID NO: 25.

In certain embodiments, the instant disclosure provides an isolated antibody that specifically binds to ApoC3 (e.g., human or cynomolgus ApoC3), comprising a light chain variable region comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to the amino acid sequence set forth in SEQ ID NO: 27, 28, 29, 30, 31, 32, or 33. In certain embodiments, the antibody comprises a light chain variable region comprising the amino acid sequence set forth in SEQ ID NO: 27, 28, 29, 30, 31, 32, or 33. In certain embodiments, the antibody comprises a light chain variable region comprising the amino acid sequence set forth in SEQ ID NO: 27. In certain embodiments, the antibody comprises a light chain variable region comprising the amino acid sequence set forth in SEQ ID NO: 28. In certain embodiments, the antibody comprises a light chain variable region comprising the amino acid sequence set forth in SEQ ID NO: 29. In certain embodiments, the antibody comprises a light chain variable region comprising the amino acid sequence set forth in SEQ ID NO: 30. In certain embodiments, the antibody comprises a light chain variable region comprising the amino acid sequence set forth in SEQ ID NO: 31. In certain embodiments, the antibody comprises a light chain variable region comprising the amino acid sequence set forth in SEQ ID NO: 32. In certain embodiments, the antibody comprises a light chain variable region comprising the amino acid sequence set forth in SEQ ID NO: 33.

In certain embodiments, the instant disclosure provides an isolated antibody that specifically binds to ApoC3 (e.g., human or cynomolgus ApoC3), comprising a heavy chain variable region comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to the amino acid sequence set forth in SEQ ID NO: 25, and a light chain variable region comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to the amino acid sequence set forth in SEQ ID NO: 27, 28, 29, 30, 31, 32, or 33. In certain embodiments, the antibody comprises a heavy chain variable region comprising the amino acid sequence set forth in SEQ ID NO: 25, and a light chain variable region comprising the amino acid sequence set forth in SEQ ID NO: 27, 28, 29, 30, 31, 32, or 33. In certain embodiments, the antibody comprises a heavy chain variable region and light chain variable region comprising the amino acid sequences set forth in SEQ ID NOs: 25 and 27, 25 and 28, 25 and 29, 25 and 30, 25 and 31, 25 and 32, or 25 and 33, respectively. In certain embodiments, the antibody comprises a heavy chain variable region and light chain variable region comprising the amino acid sequences set forth in SEQ ID NO: 25 and 27, respectively. In certain embodiments, the antibody comprises a heavy chain variable region and light chain variable region comprising the amino acid sequences set forth in SEQ ID NO: 25 and 28, respectively. In certain embodiments, the antibody comprises a heavy chain variable region and light chain variable region comprising the amino acid sequences set forth in SEQ ID NO: 25 and 29, respectively. In certain embodiments, the antibody comprises a heavy chain variable region and light chain variable region comprising the amino acid sequences set forth in SEQ ID NO: 25 and 30, respectively. In certain embodiments, the antibody comprises a heavy chain variable region and light chain variable region comprising the amino acid sequences set forth in SEQ ID NO: 25 and 31, respectively. In certain embodiments, the antibody comprises a heavy chain variable region and light chain variable region comprising the amino acid sequences set forth in SEQ ID NO: 25 and 32, respectively. In certain embodiments, the antibody comprises a heavy chain variable region and light chain variable region comprising the amino acid sequences set forth in SEQ ID NO: 25 and 33, respectively

Any Ig constant region can be used in the isolated antibodies disclosed herein. In certain embodiments, the Ig constant region is a constant region of human IgG, IgE, IgM, IgD, IgA, or IgY immunoglobulin (Ig) molecule, and/or a constant region of any class (e.g., IgG₁, IgG₂, IgG₃, IgG₄, IgA₁, and IgA₂) or any subclass (e.g., IgG₂ₐ and IgG_{2b}) of immunoglobulin molecule. In certain embodiments, the Ig constant region is a human or humanized Ig constant region.

In certain embodiments, the constant region is a variant of a wild type human Ig (e.g., IgG) heavy chain constant region, and wherein the variant human Ig heavy chain constant region has an increased affinity (e.g., increased by at least 1.5, 2, 2.5, 3, 4, 5, 6, 7, 8, 9, 10, 15, or 20 fold) for human neonatal Fc receptor (FcRn) at acidic pH (e.g., pH 5.5 to pH 6) relative to the affinity of the corresponding wild type human Ig heavy chain constant region for human FcRn under the same conditions. In certain embodiments, the variant human Ig heavy chain constant region has a similar or decreased affinity (e.g., increased by no more than 1.1, 1.2, 1.3, 1.4, 1.5, 1.6, 1.7, 1.8, 1.9, or 2 fold, equal to, or decreased) for human neonatal Fc receptor (FcRn) at physiological pH (e.g., at pH 7.4) relative to the affinity of the wild type human Ig heavy chain constant region for human FcRn under the same conditions. In certain embodiments, the constant region comprises one, two, or more amino acids (e.g., having one or more substitutions, insertions or deletions) from a wild-type Ig (e.g., IgG) constant domain or FcRn-binding fragment thereof (e.g., an Fc or hinge-Fc domain fragment). In certain embodiments, the half-life of the antibody with the variant constant region in vivo is increased relative to the half-life of the corresponding antibody with the wild-type constant domain or FcRn-binding fragment thereof in vivo. See, e.g., International Publication Nos. WO 02/060919; WO 98/23289; and WO 97/34631; and U.S. Pat. Nos. 5,869,046, 6,121,022, 6,277,375, 6,165,745, 8,088,376, and 8,163,881, all of which are herein incorporated by reference in their entireties, for examples of mutations that will increase the half-life of an antibody in vivo. In certain embodiment, the one or more different amino acids are in the second constant (CH2) domain (residues 231-340 of human IgG₁) and/or the third constant (CH3) domain (residues 341-447 of human IgG₁), numbered according to the EU numbering system. In certain embodiments, the constant region of the IgG (e.g., IgG₁, IgG₂, or IgG₄) of an antibody disclosed herein comprises the amino acids tyrosine (Y) threonine (T), and glutamic acid (E) at positions 252, 254, and 256, respectively, numbered according to the EU numbering system. See U.S. Pat. No. 7,658,921, which is herein incorporated by reference in its entirety. This type of IgG, referred to as “YTE IgG” has been shown to display fourfold increased half-life as compared to wild-type versions of the same antibody (see Dall'Acqua W F et al., (2006) J Biol Chem 281: 23514-24, which is herein incorporated by reference in its entirety). In certain embodiments, the constant region of the IgG (e.g., IgG₁) of an antibody disclosed herein comprises the amino acid alanine (A), serine (S), tyrosine (Y), or phenylalanine (F) at position 434, numbered according to the EU numbering system. In certain embodiments, the constant region of the IgG (e.g., IgG₁, IgG₂, or IgG₄) of an antibody disclosed herein comprises the amino acids lysine (K), phenylalanine (F), and tyrosine (Y) at positions 433, 434, and 436, respectively, numbered according to the EU numbering system. In certain embodiments, the constant region of the IgG (e.g., IgG₁, IgG₂, or IgG₄) of an antibody disclosed herein comprises the amino acids leucine (L) and serine (S) at positions 428 and 434, respectively, numbered according to the EU numbering system. Additional IgG constant regions that may have increased affinity to FcRn under acidic condition are described in Ward et al., Mol. Immunol. (2015) 67(200):131-41, which is herein incorporated by reference in its entirety. In certain embodiments, an antibody comprises an IgG constant domain comprising one, two, three or more amino acid substitutions of amino acid residues at positions 251-257, 285-290, 308-314, 385-389, and 428-436, numbered according to the EU numbering system.

In certain embodiments, the instant disclosure provides an isolated antibody that specifically binds to ApoC3 (e.g., human or cynomolgus ApoC3), the antibody comprising a heavy chain comprising the amino acid sequence set forth in SEQ ID NO: 34, 35, 36, 37, 38, 39, 40, 41, 42, 43, 44, 45, 46, 47, 48, or 49.

In certain embodiments, the instant disclosure provides an isolated antibody that specifically binds to ApoC3 (e.g., human or cynomolgus ApoC3), the antibody comprising a light chain comprising the amino acid sequence set forth in SEQ ID NO: 50, 51, 52, 53, 54, 55, or 56.

In certain embodiments, the instant disclosure provides an isolated antibody that specifically binds to ApoC3, the antibody comprising a heavy chain and a light chain, wherein the amino acid sequences of the heavy chain and the light chain, respectively, comprise the amino acid sequences set forth in SEQ ID NOs: 34 and 50, 35 and 50, 36 and 50, 37 and 50, 38 and 50, 39 and 50, 40 and 50, 41 and 50, 42 and 50, 43 and 50, 44 and 50, 45 and 50, 46 and 50, 47 and 50, 48 and 50, or 49 and 50.

In certain embodiments, the instant disclosure provides an isolated antibody that specifically binds to ApoC3, the antibody comprising a heavy chain and a light chain, wherein the amino acid sequences of the heavy chain and the light chain, respectively, consist of the amino acid sequences set forth in SEQ ID NOs: 34 and 50, 35 and 50, 36 and 50, 37 and 50, 38 and 50, 39 and 50, 40 and 50, 41 and 50, 42 and 50, 43 and 50, 44 and 50, 45 and 50, 46 and 50, 47 and 50, 48 and 50, or 49 and 50.

In certain embodiments, the instant disclosure provides an isolated antibody that specifically binds to ApoC3 (e.g., human or cynomolgus ApoC3), the antibody comprising a heavy chain and a light chain, wherein: the amino acid sequence of the heavy chain comprises or consists of the amino acid sequence set forth in SEQ ID NO: 34, 35, 36, 37, 38, 39, 40, 41, 42, 43, 44, 45, 46, 47, 48, or 49; and the amino acid sequence of the light chain comprises or consists of the amino acid sequence set forth in SEQ ID NO: 51.

In certain embodiments, the instant disclosure provides an isolated antibody that specifically binds to ApoC3 (e.g., human or cynomolgus ApoC3), the antibody comprising a heavy chain and a light chain, wherein: the amino acid sequence of the heavy chain comprises or consists of the amino acid sequence set forth in SEQ ID NO: 34, 35, 36, 37, 38, 39, 40, 41, 42, 43, 44, 45, 46, 47, 48, or 49; and the amino acid sequence of the light chain comprises or consists of the amino acid sequence set forth in SEQ ID NO: 52.

In certain embodiments, the instant disclosure provides an isolated antibody that specifically binds to ApoC3 (e.g., human or cynomolgus ApoC3), the antibody comprising a heavy chain and a light chain, wherein: the amino acid sequence of the heavy chain comprises or consists of the amino acid sequence set forth in SEQ ID NO: 34, 35, 36, 37, 38, 39, 40, 41, 42, 43, 44, 45, 46, 47, 48, or 49; and the amino acid sequence of the light chain comprises or consists of the amino acid sequence set forth in SEQ ID NO: 53.

In certain embodiments, the instant disclosure provides an isolated antibody that specifically binds to ApoC3 (e.g., human or cynomolgus ApoC3), the antibody comprising a heavy chain and a light chain, wherein: the amino acid sequence of the heavy chain comprises or consists of the amino acid sequence set forth in SEQ ID NO: 34, 35, 36, 37, 38, 39, 40, 41, 42, 43, 44, 45, 46, 47, 48, or 49; and the amino acid sequence of the light chain comprises or consists of the amino acid sequence set forth in SEQ ID NO: 54.

In certain embodiments, the instant disclosure provides an isolated antibody that specifically binds to ApoC3 (e.g., human or cynomolgus ApoC3), the antibody comprising a heavy chain and a light chain, wherein: the amino acid sequence of the heavy chain comprises or consists of the amino acid sequence set forth in SEQ ID NO: 34, 35, 36, 37, 38, 39, 40, 41, 42, 43, 44, 45, 46, 47, 48, or 49; and the amino acid sequence of the light chain comprises or consists of the amino acid sequence set forth in SEQ ID NO: 55.

In certain embodiments, the instant disclosure provides an isolated antibody that specifically binds to ApoC3 (e.g., human or cynomolgus ApoC3), the antibody comprising a heavy chain and a light chain, wherein: the amino acid sequence of the heavy chain comprises or consists of the amino acid sequence set forth in SEQ ID NO: 34, 35, 36, 37, 38, 39, 40, 41, 42, 43, 44, 45, 46, 47, 48, or 49; and the amino acid sequence of the light chain comprises or consists of the amino acid sequence set forth in SEQ ID NO: 56.

In certain embodiments, the isolated antibodies disclosed herein attenuate the ability of ApoC3 to inhibit hepatocyte uptake of TRL (e.g., VLDL) or TRL remnants (in vivo or in vitro). In certain embodiments, the isolated antibodies disclosed herein attenuate the ability of ApoC3 to inhibit hepatocyte uptake of TRL (e.g., VLDL) or TRL remnants by at least 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods disclosed herein or by methods known to one of skill in the art. In certain embodiments, the isolated antibodies disclosed herein attenuate the ability of ApoC3 to inhibit hepatocyte uptake of TRL (e.g., VLDL) or TRL remnants by at least about 1.1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold, as assessed by methods disclosed herein or by methods known to one of skill in the art.

In certain embodiments, the isolated antibodies disclosed herein are capable of inhibiting post-prandial lipemia in a subject when administered to the subject prior to, during, or after a meal. In certain embodiments, the anti-ApoC3 antibodies disclosed herein are capable of inhibiting post-prandial lipemia in the subject by at least 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods disclosed herein or by methods known to one of skill in the art. In certain embodiments, the anti-ApoC3 antibodies disclosed herein are capable of inhibiting post-prandial lipemia in the subject by at least about 1.1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold, as assessed by methods disclosed herein or by methods known to one of skill in the art.

In certain embodiments, the isolated antibodies disclosed herein are capable of reducing the levels of post-prandial chylomicron or chylomicron remnants in a subject when administered to the subject prior to, during, or after a meal. In certain embodiments, the anti-ApoC3 antibodies disclosed herein are capable of reducing the levels of post-prandial chylomicron or chylomicron remnants in a subject by at least 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods disclosed herein or by methods known to one of skill in the art. In certain embodiments, the anti-ApoC3 antibodies disclosed herein are capable of reducing the levels of post-prandial chylomicron or chylomicron remnants in a subject by at least about 1.1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold, as assessed by methods disclosed herein or by methods known to one of skill in the art.

In certain embodiments, the isolated antibodies disclosed herein are capable of increasing the rates of clearance of ApoC3 from the blood in a subject. In certain embodiments, the anti-ApoC3 antibodies are capable of increasing the rates of clearance of ApoC3 from the blood in a subject by at least 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods disclosed herein or by methods known to one of skill in the art. In certain embodiments, the anti-ApoC3 antibodies disclosed herein are capable of increasing the rates of clearance of ApoC3 from the blood in a subject by at least about 1.1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold, as assessed by methods disclosed herein or by methods known to one of skill in the art. Methods for assessing the clearance of ApoC3 include without limitation the isotope tracer techniques, wherein the isotope can be either radioactive or stable.

In certain embodiments, the isolated antibodies disclosed herein are capable of reducing the levels of ApoC3 in the blood in a subject. In certain embodiments, the anti-ApoC3 antibodies are capable of reducing the levels of ApoC3 in the blood in a subject by at least 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods disclosed herein or by methods known to one of skill in the art. In certain embodiments, the anti-ApoC3 antibodies disclosed herein are capable of reducing the levels of ApoC3 in the blood in a subject by at least about 1.1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold, as assessed by methods disclosed herein or by methods known to one of skill in the art. In certain embodiments, the reduction in the levels of ApoC3 in the blood in the subject is maintained for at least 1, 2, 3, 4, 5, 6, 7, 8, 9, 10, 15, 20, 25, 30, 35, 40, 45, or 50 days, or at least 1, 2, 3, 4, 5, 6, 7, or 8 weeks.

In certain embodiments, the isolated antibodies disclosed herein are capable of binding to lipid-bound ApoC3 (e.g., ApoC3 bound to triglyceride, TRL (e.g., VLDL) or TRL remnants). In certain embodiments, the isolated antibodies disclosed herein do not inhibit the binding of ApoC3 to a lipid or a lipoprotein. In certain embodiments, the antibodies disclosed herein do not compete for the binding of ApoC3 with a lipid or a lipoprotein. In certain embodiments, the lipid comprises a fatty acid chain. In certain embodiments, the lipid comprises a phosphatidyl group. In certain embodiments, the lipid comprises a phosphatidylcholine (e.g., DMPC), a phosphatidylserine, a phosphatidylethanolamine, a phosphatidylinositol or a phosphatidylglycerol. In certain embodiments, the lipid is a triglyceride. In certain embodiments, the lipoprotein is a TRL (e.g., VLDL) or a TRL remnant. In certain embodiments, the ability of ApoC3 to bind to lipids and lipoproteins (e.g., triglyceride, TRL (e.g., VLDL) or TRL remnants) in the presence of an anti-ApoC3 antibody disclosed herein is at least 50%, 55%, 60%, 65%, 70%, 75%, 80%, 85%, 90%, 95%, 98%, or 99% of the ability of ApoC3 to bind to the same lipids and lipoproteins in the absence of an anti-ApoC3 antibody, as assessed by methods disclosed herein or by methods known to one of skill in the art.

In certain embodiments, the isolated antibodies disclosed herein attenuate the ability of ApoC3 to inhibit hepatocyte uptake of TRL (e.g., VLDL) or TRL remnants. In certain embodiments, the uptake of TRL (e.g., VLDL) or TRL remnants by hepatocytes (e.g., HepG2 cells) in the presence of an anti-ApoC3 antibody as disclosed herein is at least 1.1, 1.2, 1.3, 1.4, 1.5, 1.6, 1.7, 1.8, 1.9, 2, 2.5, 3, 3.5, 4, 4.5, or 5 folds higher than the uptake of TRL (e.g., VLDL) or TRL remnants by hepatocytes (e.g., HepG2 cells) in the absence of an anti-ApoC3 antibody.

In certain embodiments, the isolated antibodies disclosed herein attenuate the ability of ApoC3 to inhibit hepatocyte uptake of TRL (e.g., VLDL) or TRL remnants, and are capable of binding to lipid-bound ApoC3 (e.g., ApoC3 bound to triglyceride, TRL (e.g., VLDL) or TRL remnants.

### 3. Methods of Use

ApoC3 inhibits TRL (e.g., VLDL) and TRL remnant uptake and clearance by hepatocytes and inhibits lipoprotein lipase-mediated lipolysis of TRL (e.g., VLDL), thereby functioning to increase triglyceride levels in the blood of a subject. In certain embodiments, the anti-ApoC3 antibodies disclosed herein can attenuate the ability of ApoC3 to inhibit TRL (e.g., VLDL) and TRL remnant uptake and clearance by hepatocytes or attenuate the ability of ApoC3 to inhibit lipoprotein lipase-mediated lipolysis of TRL (e.g., VLDL). Accordingly, in certain embodiments, the instant disclosure provides a method for inhibiting the activity of ApoC3 in the blood of a subject, the method comprising administering to the subject an effective amount of an anti-ApoC3 antibody or pharmaceutical composition disclosed herein. In certain embodiments, the activity of ApoC3 is inhibition of TRL (e.g., VLDL) and TRL remnants uptake and clearance by hepatocytes. In certain embodiments, the activity of ApoC3 is inhibition of lipoprotein lipase-mediated lipolysis of TRL. In certain embodiments, the activity of ApoC3 is inhibition of TRL (e.g., VLDL) and TRL remnants uptake and clearance by hepatocytes and inhibition of lipoprotein lipase-mediated lipolysis of TRL.

The anti-ApoC3 antibodies disclosed herein are useful for increasing the rate of clearance of ApoC3 from the blood in a subject. Accordingly, in certain embodiments, the instant disclosure provides a method for increasing the rate of clearance of ApoC3 from the blood in a subject, the method comprising administering to the subject an effective amount of an anti-ApoC3 antibody or pharmaceutical composition disclosed herein.

The anti-ApoC3 antibodies disclosed herein are useful for reducing the level of ApoC3 in the blood of a subject. Accordingly, in certain embodiments, the instant disclosure provides a method for reducing the level of ApoC3 in the blood of a subject, the method comprising administering to the subject an effective amount of an anti-ApoC3 antibody or pharmaceutical composition disclosed herein. In certain embodiments, the method reduces the level of ApoC3 in the blood of a subject by at least 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods disclosed herein or by methods known to one of skill in the art. In certain embodiments, the method reduces the levels of ApoC3 in the blood in a subject by at least about 1.1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold, as assessed by methods disclosed herein or by methods known to one of skill in the art. In certain embodiments, the reduction in the levels of ApoC3 in the blood in the subject is maintained for at least 1, 2, 3, 4, 5, 6, 7, 8, 9, 10, 15, 20, 25, 30, 35, 40, 45, or 50 days, or at least 1, 2, 3, 4, 5, 6, 7, or 8 weeks.

The anti-ApoC3 antibodies disclosed herein are useful for reducing triglyceride levels in the blood of a subject. Accordingly, in certain embodiments, the instant disclosure provides a method for reducing triglyceride levels in the blood of a subject, the method comprising administering to the subject an effective amount of an anti-ApoC3 antibody or pharmaceutical composition disclosed herein.

The anti-ApoC3 antibodies disclosed herein are useful for the treatment of hypertriglyceridemia. Accordingly, in certain embodiments, the instant disclosure provides a method for treating hypertriglyceridemia in a subject, the method comprising administering to the subject an effective amount of an anti-ApoC3 antibody or pharmaceutical composition disclosed herein. In certain embodiments, the instant disclosure provides a method for treating chylomicronemia in a subject, the method comprising administering to the subject an effective amount of an anti-ApoC3 antibody or pharmaceutical composition disclosed herein. In certain embodiments, the instant disclosure provides a method for treating chylomicronemia syndrome in a subject, the method comprising administering to the subject an effective amount of an anti-ApoC3 antibody or pharmaceutical composition disclosed herein.

The anti-ApoC3 antibodies disclosed herein are useful for the treatment and prevention of post-prandial lipemia in a subject. Accordingly, in certain embodiments, the instant disclosure provides a method for inhibiting post-prandial lipemia in a subject, the method comprising administering to the subject an effective amount of an anti-ApoC3 antibody or pharmaceutical composition disclosed herein. The anti-ApoC3 antibody can be administered to the subject prior to, during, or after a meal.

Without wishing to be bound by theory, Applicants believe that, in certain embodiments, the antibodies disclosed herein are capable of reducing the levels of post-prandial chylomicron or chylomicron remnants in a subject when administered to the subject prior to, during, or after a meal. Accordingly, in certain embodiments, the instant disclosure provides a method for reducing the levels of post-prandial chylomicron or chylomicron remnants in a subject, the method comprising administering to the subject an effective amount of an anti-ApoC3 antibody or pharmaceutical composition disclosed herein. The anti-ApoC3 antibody can be administered to the subject prior to, during, or after a meal.

The reduction of triglyceride levels in blood in patients with hypertriglyceridemia may reduce the risk of development of pancreatitis. Accordingly, in certain embodiments, the instant disclosure provides a method for reducing the risk of pancreatitis in a subject with hypertriglyceridemia, the method comprising administering to the subject an effective amount of an anti-ApoC3 antibody or pharmaceutical composition disclosed herein

The anti-ApoC3 antibodies disclosed herein are useful for reducing the risk of cardiovascular disease in a subject. Accordingly, in certain embodiments, the instant disclosure provides a method for reducing the risk of cardiovascular disease in a subject with hypertriglyceridemia, the method comprising administering to the subject an effective amount of an anti-ApoC3 antibody or pharmaceutical composition disclosed herein. The risk of developing any cardiovascular disease associated with or caused by hypertriglyceridemia or excessive post prandial lipemia can be reduced by administration of an anti-ApoC3 antibody or pharmaceutical composition disclosed herein. Cardiovascular disease for which the risk can be reduced include without limitation coronary artery disease, atherosclerosis, angina, myocardial infarction, and stroke.

The anti-ApoC3 antibodies or pharmaceutical compositions disclosed herein can be administered either alone or in combination an additional therapeutic agent. In certain embodiments, the additional therapeutic agent is another lipid lowering agent. Any one or more lipid lowering agent can be used in combination with an anti-ApoC3 antibody or pharmaceutical composition disclosed herein. Suitable lipid lowering agents include without limitation HMG-CoA reductase inhibitors (e.g., atorvastatin, fluvastatin, lovastatin, pitavastatin, pravastatin, rosuvastatin or simvastatin), fibrates, niacin, bile acid sequestrants (e.g., cholestyramine, colestipol, and colesevelam), inhibitors of dietary cholesterol absorption (e.g., ezetimibe), microsomal triglyceride transfer protein (MTP) inhibitors (e.g., lomitapide), phytosterols, pancreatic lipase inhibitors (e.g., orlistat), cholesteryl ester transfer protein inhibitors, squalene synthase inhibitors (e.g., TAK-475, zaragozic acid, and RPR 107393), ApoA-1 Milano, succinobucol (AGI-1067), Apoprotein-B inhibitors (e.g., Mipomersen), and proprotein convertase subtilisin/kexin type 9 (PCSK9) inhibitors (e.g., alirocumab, evolocumab, and bococizumab). In certain embodiments, the additional lipid lowering agent is a combination of ezetimibe and an HMG-CoA reductase inhibitor. In certain embodiments, the lipid lowering agent is a combination of ezetimibe, an HMG-CoA reductase inhibitor, and a PCSK9 inhibitor.

The anti-ApoC3 antibodies or pharmaceutical compositions disclosed herein may be delivered to a subject by a variety of routes. These include, but are not limited to, parenteral, intradermal, intramuscular, intraperitoneal, intravenous, and subcutaneous routes. In certain embodiments, the antibody or pharmaceutical composition disclosed herein is delivered subcutaneously or intravenously.

The amount of an anti-ApoC3 antibody or pharmaceutical composition disclosed herein which will be effective in the treatment or prevention of a condition will depend on the nature of the disease, and can be empirically determined by standard clinical techniques. The precise dose to be employed in a composition will also depend on the route of administration, and the seriousness of the infection or disease caused by it, and should be decided according to the judgment of the practitioner and each subject's circumstances. For example, effective doses may also vary depending upon means of administration, target site, physiological state of the patient (including age, body weight and health), whether the patient is human or an animal, other medications administered, or whether treatment is prophylactic or therapeutic. The anti-ApoC3 antibodies or pharmaceutical compositions disclosed herein can be administered at any frequency (e.g., about every week, every two weeks, every three weeks, every four weeks, every month, or every two months). Usually, the patient is a human, but non-human mammals including transgenic mammals can also be treated. Treatment dosages and regimens are optimally titrated to optimize safety and efficacy.

The anti-ApoC3 antibodies disclosed herein can also be used to assay ApoC3 (e.g., human or cynomolgus ApoC3) protein levels in a biological sample using classical immunohistological methods known to those of skill in the art, including immunoassays, such as the enzyme linked immunosorbent assay (ELISA), immunoprecipitation, or Western blotting. Suitable antibody assay labels are known in the art and include enzyme labels, such as, glucose oxidase; radioisotopes, such as iodine (¹²⁵I, ¹²¹I) carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labels can be used to label an antibody disclosed herein. Alternatively, a second antibody that recognizes an anti-ApoC3 antibody disclosed herein can be labeled and used in combination with an anti-ApoC3 antibody to detect ApoC3 (e.g., human or cynomolgus ApoC3) protein levels.

Assaying for the expression level of ApoC3 (e.g., human or cynomolgus ApoC3) protein is intended to include qualitatively or quantitatively measuring or estimating the level of ApoC3 (e.g., human or cynomolgus ApoC3) protein in a first biological sample either directly (e.g., by determining or estimating absolute protein level) or relatively (e.g., by comparing to the disease associated protein level in a second biological sample). ApoC3 (e.g., human or cynomolgus ApoC3) polypeptide expression level in the first biological sample can be measured or estimated and compared to a standard ApoC3 (e.g., human or cynomolgus ApoC3) protein level, the standard being taken from a second biological sample obtained from an individual not having the disorder or being determined by averaging levels from a population of individuals not having the disorder. As will be appreciated in the art, once the “standard” ApoC3 (e.g., human or cynomolgus ApoC3) polypeptide level is known, it can be used repeatedly as a standard for comparison.

As used herein, the term “biological sample” refers to any biological sample obtained from a subject, cell line, tissue, or other source of cells potentially expressing ApoC3 (e.g., human or cynomolgus ApoC3). Methods for obtaining tissue biopsies and body fluids from animals (e.g., humans) are well known in the art. Biological samples include peripheral mononuclear blood cells.

The anti-ApoC3 antibodies disclosed herein can be used for prognostic, diagnostic, monitoring and screening applications, including in vitro and in vivo applications well known and standard to the skilled artisan and based on the present description. Prognostic, diagnostic, monitoring and screening assays and kits for in vitro assessment and evaluation of immune system status or immune response may be utilized to predict, diagnose and monitor to evaluate patient samples including those known to have or suspected of having elevated ApoC3 activity. In one embodiment, an anti-ApoC3 antibody can be used in immunohistochemistry of biopsy samples. In another embodiment, an anti-ApoC3 antibody can be used to detect levels of ApoC3 (e.g., human or cynomolgus ApoC3), which levels can then be linked to certain disease symptoms. Anti-ApoC3 antibodies disclosed herein may carry a detectable or functional label. When fluorescence labels are used, currently available microscopy and fluorescence-activated cell sorter analysis (FACS) or combination of both methods procedures known in the art may be utilized to identify and to quantitate the specific binding members. Anti-ApoC3 antibodies disclosed herein may carry a fluorescence label. Exemplary fluorescence labels include, for example, reactive and conjugated probes e.g. Aminocoumarin, Fluorescein and Texas red, Alexa Fluor dyes, Cy dyes and DyLight dyes. An anti-ApoC3 antibody may carry a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac and ¹⁸⁶Re. When radioactive labels are used, currently available counting procedures known in the art may be utilized to identify and quantitate the specific binding of anti-ApoC3 antibody to ApoC3 (e.g., human or cynomolgus ApoC3). In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an anti-ApoC3 antibody under conditions that allow for the formation of a complex between the antibody and ApoC3 (e.g., human or cynomolgus ApoC3). Any complexes formed between the antibody and ApoC3 (e.g., human or cynomolgus ApoC3) are detected and compared in the sample and the control. The antibodies disclosed herein can also be used to purify ApoC3 (e.g., human or cynomolgus ApoC3) via immunoaffinity purification. Also included herein is an assay system which may be prepared in the form of a test kit for the quantitative analysis of the extent of the presence of, for instance, ApoC3 (e.g., human or cynomolgus ApoC3). The system or test kit may comprise a labeled component, e.g., a labeled ApoC3 antibody, and one or more additional immunochemical reagents.

### 4. Pharmaceutical Compositions

Provided herein are pharmaceutical compositions comprising an anti-ApoC3 antibody disclosed herein having the desired degree of purity in a physiologically acceptable carrier, excipient or stabilizer (Remington's Pharmaceutical Sciences (1990) Mack Publishing Co., Easton, Pa.). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); or non-ionic surfactants such as TWEEN™, PLURONICS™ or polyethylene glycol (PEG).

In a specific embodiment, pharmaceutical compositions comprise an anti-ApoC3 antibody disclosed herein, and optionally one or more additional prophylactic or therapeutic agents, in a pharmaceutically acceptable carrier. In a specific embodiment, pharmaceutical compositions comprise an effective amount of an antibody disclosed herein, and optionally one or more additional prophylactic or therapeutic agents, in a pharmaceutically acceptable carrier. In some embodiments, the antibody is the only active ingredient included in the pharmaceutical composition. Pharmaceutical compositions disclosed herein can be useful in inhibiting, ApoC3 activity and treating a condition, such as cancer or an infectious disease.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition may be formulated for any route of administration to a subject. Specific examples of routes of administration include intranasal, oral, pulmonary, transdermal, intradermal, and parenteral. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Preparations for parenteral administration of an antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

An anti-ApoC3 antibody disclosed herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microtine powder for insufflations, alone or in combination with an inert carrier such as lactose. In such a case, the particles of the formulation will, in one embodiment, have diameters of less than 50 microns, in one embodiment less than 10 microns.

An anti-ApoC3 antibody disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

In certain embodiments, a pharmaceutical composition comprising an anti-ApoC3 antibody disclosed herein is a lyophilized powder, which can be reconstituted for administration as solutions, emulsions and other mixtures. It may also be reconstituted and formulated as solids or gels. The lyophilized powder is prepared by dissolving an antibody disclosed herein, or a pharmaceutically acceptable derivative thereof, in a suitable solvent. In some embodiments, the lyophilized powder is sterile. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

The anti-ApoC3 antibodies disclosed herein and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

The compositions to be used for in vivo administration can be sterile. This is readily accomplished by filtration through, e.g., sterile filtration membranes.

### 5. Polynucleotides, Vectors and Methods of Producing Anti-ApoC3 Antibodies

In another aspect, provided herein are polynucleotides comprising a nucleotide sequence encoding an anti-ApoC3 antibody disclosed herein (e.g., a light chain variable region or heavy chain variable region), and vectors, e.g., vectors comprising such polynucleotides for recombinant expression in host cells (e.g., E. coli and mammalian cells).

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors or other chemicals. In a specific embodiment, a nucleic acid molecule(s) encoding an antibody disclosed herein is isolated or purified.

In particular aspects, provided herein are polynucleotides comprising nucleotide sequences encoding antibodies, which specifically bind to ApoC3 (e.g., human or cynomolgus ApoC3) polypeptide and comprises an amino acid sequence as disclosed herein, as well as antibodies which compete with such antibodies for binding to ApoC3 (e.g., human or cynomolgus ApoC3) polypeptide (e.g., in a dose-dependent manner), or which binds to the same epitope as that of such antibodies.

In certain aspects, provided herein are polynucleotides comprising a nucleotide sequence encoding the light chain or heavy chain of an antibody disclosed herein. The polynucleotides can comprise nucleotide sequences encoding the VH, VL or CDRs of antibodies disclosed herein (see, e.g., Tables 1-4 herein).

Also provided herein are polynucleotides encoding an anti-ApoC3 antibody that are optimized, e.g., by codon/RNA optimization, replacement with heterologous signal sequences, and elimination of mRNA instability elements. Methods to generate optimized nucleic acids encoding an anti-ApoC3 antibody (e.g., light chain, heavy chain, VH domain, or VL domain) for recombinant expression by introducing codon changes or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties or function as the original amino acid. Such methods can increase expression of an anti-ApoC3 by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an anti-ApoC3 antibody encoded by polynucleotides that have not been optimized.

In certain embodiments, an optimized polynucleotide sequence encoding an anti-ApoC3 antibody disclosed herein (e.g., VL domain or VH domain) can hybridize to an antisense (e.g., complementary) polynucleotide of an unoptimized polynucleotide sequence encoding an anti-ApoC3 antibody disclosed herein (e.g., VL domain or VH domain). In specific embodiments, an optimized nucleotide sequence encoding an anti-ApoC3 antibody disclosed herein or a fragment hybridizes under high stringency conditions to antisense polynucleotide of an unoptimized polynucleotide sequence encoding an anti-ApoC3 antibody disclosed herein. In a specific embodiment, an optimized nucleotide sequence encoding an anti-ApoC3 antibody disclosed herein hybridizes under high stringency, intermediate or lower stringency hybridization conditions to an antisense polynucleotide of an unoptimized nucleotide sequence encoding an anti-ApoC3 antibody disclosed herein. Information regarding hybridization conditions has been described, see, e.g., U.S. Patent Application Publication No. US 2005/0048549 (e.g., paragraphs 72-73), which is incorporated herein by reference.

The polynucleotides can be obtained, and the nucleotide sequence of the polynucleotides determined, by any method known in the art. Nucleotide sequences encoding antibodies disclosed herein, e.g., antibodies described in Tables 1-5, and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17: 242-6), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody disclosed herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain or heavy chain of an antibody. Such PCR amplification methods can be used to obtain nucleic acids comprising the sequence encoding the variable light chain region or the variable heavy chain region of an antibody. The amplified nucleic acids can be cloned into vectors for expression in host cells and for further cloning, for example, to generate chimeric and humanized antibodies.

If a clone containing a nucleic acid encoding a particular antibody is not available, but the sequence of the antibody molecule is known, a nucleic acid encoding the immunoglobulin can be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody disclosed herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding anti-ApoC3 (e.g., human or cynomolgus ApoC3) antibodies disclosed herein can be readily isolated and sequenced using conventional procedures (e.g., by using oligonucleotide probes that are capable of binding specifically to genes encoding the heavy and light chains of the anti-ApoC3 (e.g., human or cynomolgus ApoC3) antibodies). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-ApoC3 (e.g., human or cynomolgus ApoC3) antibodies in the recombinant host cells.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable region, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, for example, by substituting the coding sequence for human heavy and light chain constant domains in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody disclosed herein. In specific embodiments, polynucleotides disclosed herein hybridize under high stringency, intermediate or lower stringency hybridization conditions to polynucleotides encoding a VH domain or VL domain provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3.

In certain aspects, provided herein are cells (e.g., host cells) expressing (e.g., recombinantly) antibodies disclosed herein which specifically bind to ApoC3 (e.g., human or cynomolgus ApoC3) and related polynucleotides and expression vectors. Provided herein are vectors (e.g., expression vectors) comprising polynucleotides comprising nucleotide sequences encoding anti-ApoC3 (e.g., human or cynomolgus ApoC3) antibodies or a fragment for recombinant expression in host cells, preferably in mammalian cells. Also provided herein are host cells comprising such vectors for recombinantly expressing anti-ApoC3 (e.g., human or cynomolgus ApoC3) antibodies disclosed herein (e.g., human or humanized antibody). In a particular aspect, provided herein are methods for producing an antibody disclosed herein, comprising expressing such antibody from a host cell.

Recombinant expression of an antibody disclosed herein (e.g., a full-length antibody, heavy or light chain of an antibody, or a single chain antibody disclosed herein) that specifically binds to ApoC3 (e.g., human or cynomolgus ApoC3) involves construction of an expression vector containing a polynucleotide that encodes the antibody. Once a polynucleotide encoding an antibody molecule, heavy or light chain of an antibody, (e.g., heavy or light chain variable regions) disclosed herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are disclosed herein. Methods which are well known to those skilled in the art can be used to construct expression vectors containing antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule disclosed herein, a heavy or light chain of an antibody, a heavy or light chain variable region of an antibody, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464) and variable regions of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody disclosed herein. Thus, provided herein are host cells containing a polynucleotide encoding an antibody disclosed herein, or a heavy or light chain thereof, or fragment thereof, or a single chain antibody disclosed herein, operably linked to a promoter for expression of such sequences in the host cell. In certain embodiments, for the expression of double-chained antibodies, vectors encoding both the heavy and light chains, individually, can be co-expressed in the host cell for expression of the entire immunoglobulin molecule, as detailed below. In certain embodiments, a host cell contains a vector comprising a polynucleotide encoding both the heavy chain and light chain of an antibody disclosed herein. In specific embodiments, a host cell contains two different vectors, a first vector comprising a polynucleotide encoding a heavy chain or a heavy chain variable region of an antibody disclosed herein, or a fragment thereof, and a second vector comprising a polynucleotide encoding a light chain or a light chain variable region of an antibody disclosed herein, or a fragment thereof. In other embodiments, a first host cell comprises a first vector comprising a polynucleotide encoding a heavy chain or a heavy chain variable region of an antibody disclosed herein, or a fragment thereof, and a second host cell comprises a second vector comprising a polynucleotide encoding a light chain or a light chain variable region of an antibody disclosed herein. In specific embodiments, a heavy chain/heavy chain variable region expressed by a first cell associated with a light chain/light chain variable region of a second cell to form an anti-ApoC3 antibody disclosed herein. In certain embodiments, provided herein is a population of host cells comprising such first host cell and such second host cell.

In a particular embodiment, provided herein is a population of vectors comprising a first vector comprising a polynucleotide encoding a light chain/light chain variable region of an anti-ApoC3 antibody disclosed herein, and a second vector comprising a polynucleotide encoding a heavy chain/heavy chain variable region of an anti-ApoC3 antibody disclosed herein.

A variety of host-expression vector systems can be utilized to express antibody molecules disclosed herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule disclosed herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies disclosed herein are CHO cells, for example CHO cells from the CHO GS System™ (Lonza). In a particular embodiment, cells for expressing antibodies disclosed herein are human cells, e.g., human cell lines. In a specific embodiment, a mammalian expression vector is pOptiVEC™, pcDNA3.3, or pCDNA3.1Neo. In a particular embodiment, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells, in conjunction with a vector such as the major intermediate early gene promoter element from human cytomegalovirus is an effective expression system for antibodies (Foecking M K & Hofstetter H (1986) Gene 45: 101-5; and Cockett M I et al., (1990) Biotechnology 8(7): 662-7). In certain embodiments, antibodies disclosed herein are produced by CHO cells or NS0 cells. In a specific embodiment, the expression of nucleotide sequences encoding antibodies disclosed herein which specifically bind ApoC3 (e.g., human or cynomolgus ApoC3) is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the antibody coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region El or E3) will result in a recombinant virus that is viable and capable of expressing the antibody molecule in infected hosts (e.g., see Logan J & Shenk T (1984) PNAS 81(12): 3655-9). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153: 516-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT2O and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In certain embodiments, anti-ApoC3 (e.g., human or cynomolgus ApoC3) antibodies disclosed herein are produced in mammalian cells, such as CHO cells.

In a specific embodiment, the antibodies disclosed herein have reduced fucose content or no fucose content. Such antibodies can be produced using techniques known one skilled in the art. For example, the antibodies can be expressed in cells deficient or lacking the ability of to fucosylate. In a specific example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express an anti-ApoC3 antibody disclosed herein can be engineered. In specific embodiments, a cell provided herein stably expresses a light chain/light chain variable region and a heavy chain/heavy chain variable region which associate to form an antibody disclosed herein.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-ApoC3 antibody disclosed herein. Such engineered cell lines can be particularly useful in screening and evaluation of compositions that interact directly or indirectly with the antibody molecule.

A number of selection systems can be used, including but not limited to the herpes simplex virus thymidine kinase (Wigler M et al., (1977) Cell 11(1): 223-32), hypoxanthineguanine phosphoribosyltransferase (Szybalska E H & Szybalski W (1962) PNAS 48(12): 2026-2034) and adenine phosphoribosyltransferase (Lowy I et al., (1980) Cell 22(3): 817-23) genes in tk-, hgprt- or aprt-cells, respectively. Also, antimetabolite resistance can be used as the basis of selection for the following genes: dhfr, which confers resistance to methotrexate (Wigler M et al., (1980) PNAS 77(6): 3567-70; O'Hare K et al., (1981) PNAS 78: 1527-31); gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Felgner P L (1993) Trends Biotechnol 11(5): 211-5); and hygro, which confers resistance to hygromycin (Santerre R F et al., (1984) Gene 30(1-3): 147-56). Methods commonly known in the art of recombinant DNA technology can be routinely applied to select the desired recombinant clone and such methods are described, for example, in Ausubel F M et al., (eds.), Current Protocols in Molecular Biology, John Wiley & Sons, N Y (1993); Kriegler M, Gene Transfer and Expression, A Laboratory Manual, Stockton Press, N Y (1990); and in Chapters 12 and 13, Dracopoli N C et al., (eds.), Current Protocols in Human Genetics, John Wiley & Sons, N Y (1994); Colbere-Garapin F et al., (1981) J Mol Biol 150: 1-14, which are incorporated by reference herein in their entireties.

The expression levels of an antibody molecule can be increased by vector amplification (for a review, see Bebbington C R & Hentschel CCG, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse G F et al., (1983) Mol Cell Biol 3: 257-66).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot N J (1986) Nature 322: 562-565; and Köhler G (1980) PNAS 77: 2197-2199). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody disclosed herein), and a second gene and (e.g., light chain of an antibody disclosed herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule disclosed herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies disclosed herein can be fused to heterologous polypeptide sequences disclosed herein or otherwise known in the art to facilitate purification.

In specific embodiments, an antibody disclosed herein is isolated or purified. Generally, an isolated antibody is one that is substantially free of other antibodies with different antigenic specificities than the isolated antibody. For example, in a particular embodiment, a preparation of an antibody disclosed herein is substantially free of cellular material or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly, such preparations of the antibody have less than about 30%, 20%, 10%, or 5% (by dry weight) of chemical precursors or compounds other than the antibody of interest. In a specific embodiment, antibodies disclosed herein are isolated or purified.

Antibodies that specifically bind to ApoC3 (e.g., human or cynomolgus ApoC3) can be produced by any method known in the art for the synthesis of antibodies, for example, by chemical synthesis or by recombinant expression techniques. The methods disclosed herein employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, N.Y.; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In a specific embodiment, an antibody disclosed herein is an antibody (e.g., recombinant antibody) prepared, expressed, created or isolated by any means that involves creation, e.g., via synthesis, genetic engineering of DNA sequences. In certain embodiments, such antibody comprises sequences (e.g., DNA sequences or amino acid sequences) that do not naturally exist within the antibody germline repertoire of an animal or mammal (e.g., human) in vivo.

In one aspect, provided herein is a method of making an antibody which specifically binds to ApoC3 (e.g., human or cynomolgus ApoC3) comprising culturing a cell or host cell disclosed herein. In a certain aspect, provided herein is a method of making an antibody which specifically binds to ApoC3 (e.g., human or cynomolgus ApoC3) comprising expressing (e.g., recombinantly expressing) the antibody using a cell or host cell disclosed herein (e.g., a cell or a host cell comprising polynucleotides encoding an antibody disclosed herein). In a particular embodiment, the cell is an isolated cell. In a particular embodiment, the exogenous polynucleotides have been introduced into the cell. In a particular embodiment, the method further comprises the step of purifying the antibody obtained from the cell or host cell.

Methods for producing polyclonal antibodies are known in the art (see, for example, Chapter 11 in: Short Protocols in Molecular Biology, (2002) 5th Ed., Ausubel F M et al., eds., John Wiley and Sons, New York).

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981). The term “monoclonal antibody” as used herein is not limited to antibodies produced through hybridoma technology. For example, monoclonal antibodies can be produced recombinantly from host cells exogenously expressing an antibody disclosed herein, for example, light chain or heavy chain of such antibody.

In specific embodiments, a “monoclonal antibody,” as used herein, is an antibody produced by a single cell (e.g., hybridoma or host cell producing a recombinant antibody), wherein the antibody specifically binds to ApoC3 (e.g., human or cynomolgus ApoC3) as determined, e.g., by ELISA or other antigen-binding or competitive binding assay known in the art or in the examples provided herein. In particular embodiments, a monoclonal antibody can be a chimeric antibody or a humanized antibody. In certain embodiments, a monoclonal antibody is a monovalent antibody or multivalent (e.g., bivalent) antibody. In particular embodiments, a monoclonal antibody is a monospecific or multispecific antibody (e.g., bispecific antibody). Monoclonal antibodies disclosed herein can, for example, be made by the hybridoma method as described in Kohler G & Milstein C (1975) Nature 256: 495 or can, e.g., be isolated from phage libraries using the techniques as disclosed herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel F M et al., supra).

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the protein (e.g., ApoC3 (e.g., human or cynomolgus ApoC3)) used for immunization. Alternatively, lymphocytes may be immunized in vitro. Lymphocytes then are fused with myeloma cells using a suitable fusing agent, such as polyethylene glycol, to form a hybridoma cell (Goding J W (Ed), Monoclonal Antibodies: Principles and Practice, pp. 59-103 (Academic Press, 1986)). Additionally, a RIMMS (repetitive immunization multiple sites) technique can be used to immunize an animal (Kilpatrick K E et al., (1997) Hybridoma 16:381-9, incorporated by reference in its entirety).

In some embodiments, mice (or other animals, such as rats, monkeys, donkeys, pigs, sheep, hamster, or dogs) can be immunized with an antigen (e.g., ApoC3 (e.g., human or cynomolgus ApoC3)) and once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the American Type Culture Collection (ATCC®) (Manassas, Va.), to form hybridomas. Hybridomas are selected and cloned by limited dilution. In certain embodiments, lymph nodes of the immunized mice are harvested and fused with NS0 myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as NS0 cell line or those derived from MOPC-21 and MPC-11 mouse tumors available from the Salk Institute Cell Distribution Center, San Diego, Calif., USA, and SP-2 or X63-Ag8.653 cells available from the American Type Culture Collection, Rockville, Md., USA. Human myeloma and mouse-human heteromyeloma cell lines also have been described for the production of human monoclonal antibodies (Kozbor D (1984) J Immunol 133: 3001-5; Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against ApoC3 (e.g., human or cynomolgus ApoC3). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, or activity, the clones may be subcloned by limiting dilution procedures and grown by standard methods (Goding J W (Ed), Monoclonal Antibodies: Principles and Practice, supra). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies disclosed herein include antibody fragments which recognize specific ApoC3 (e.g., human or cynomolgus ApoC3) and can be generated by any technique known to those of skill in the art. For example, Fab and F(ab′)₂ fragments disclosed herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies disclosed herein can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies disclosed herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce antibody fragments such as Fab, Fab′ and F(ab′)2 fragments can also be employed using methods known in the art such as those disclosed in PCT publication No. WO 92/22324; Mullinax R L et al., (1992) BioTechniques 12(6): 864-9; Sawai H et al., (1995) Am J Reprod Immunol 34: 26-34; and Better M et al., (1988) Science 240: 1041-1043.

In certain embodiments, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison S L (1985) Science 229: 1202-7; Oi V T & Morrison S L (1986) BioTechniques 4: 214-221; Gillies S D et al., (1989) J Immunol Methods 125: 191-202; and U.S. Pat. Nos. 5,807,715, 4,816,567, 4,816,397, and 6,331,415.

A humanized antibody is capable of binding to a predetermined antigen and which comprises a framework region having substantially the amino acid sequence of a human immunoglobulin and CDRs having substantially the amino acid sequence of a non-human immunoglobulin (e.g., a murine immunoglobulin). In particular embodiments, a humanized antibody also comprises at least a portion of an immunoglobulin constant region (Fc), typically that of a human immunoglobulin. The antibody also can include the CH1, hinge, CH2, CH3, and CH4 regions of the heavy chain. A humanized antibody can be selected from any class of immunoglobulins, including IgM, IgG, IgD, IgA and IgE, and any isotype, including IgG₁, IgG₂, IgG₃ and IgG₄. Humanized antibodies can be produced using a variety of techniques known in the art, including but not limited to, CDR-grafting (European Patent No. EP 239400; International Publication No. WO 91/09967; and U.S. Pat. Nos. 5,225,539, 5,530,101, and 5,585,089), veneering or resurfacing (European Patent Nos. EP 592106 and EP 519596; Padlan E A (1991) Mol Immunol 28(4/5): 489-498; Studnicka G M et al., (1994) Prot Engineering 7(6): 805-814; and Roguska M A et al., (1994) PNAS 91: 969-973), chain shuffling (U.S. Pat. No. 5,565,332), and techniques disclosed in, e.g., U.S. Pat. Nos. 6,407,213, 5,766,886, International Publication No. WO 93/17105; Tan P et al., (2002) J Immunol 169: 1119-25; Caldas C et al., (2000) Protein Eng. 13(5): 353-60; Morea V et al., (2000) Methods 20(3): 267-79; Baca M et al., (1997) J Biol Chem 272(16): 10678-84; Roguska M A et al., (1996) Protein Eng 9(10): 895 904; Couto J R et al., (1995) Cancer Res. 55 (23 Supp): 5973s-5977s; Couto J R et al., (1995) Cancer Res 55(8): 1717-22; Sandhu J S (1994) Gene 150(2): 409-10 and Pedersen J T et al., (1994) J Mol Biol 235(3): 959-73. See also U.S. Application Publication No. US 2005/0042664 A1 (Feb. 24, 2005), which is incorporated by reference herein in its entirety.

Methods for making multispecific (e.g., bispecific antibodies) have been described, see, for example, U.S. Pat. Nos. 7,951,917; 7,183,076; 8,227,577; 5,837,242; 5,989,830; 5,869,620; 6,132,992 and 8,586,713.

Single domain antibodies, for example, antibodies lacking the light chains, can be produced by methods well known in the art. See Riechmann L & Muyldermans S (1999) J Immunol 231: 25-38; Nuttall S D et al., (2000) Curr Pharm Biotechnol 1(3): 253-263; Muyldermans S, (2001) J Biotechnol 74(4): 277-302; U.S. Pat. No. 6,005,079; and International Publication Nos. WO 94/04678, WO 94/25591 and WO 01/44301.

Further, antibodies that specifically bind to ApoC3 (e.g., human or cynomolgus ApoC3) can, in turn, be utilized to generate anti-idiotype antibodies that “mimic” an antigen using techniques well known to those skilled in the art. (See, e.g., Greenspan N S & Bona C A (1989) FASEB J 7(5): 437-444; and Nissinoff A (1991) J Immunol 147(8): 2429-2438).

In particular embodiments, an antibody disclosed herein, which binds to the same epitope of ApoC3 (e.g., human or cynomolgus ApoC3) as an anti-ApoC3 antibody disclosed herein, is a human antibody. In particular embodiments, an antibody disclosed herein, which competitively blocks (e.g., in a dose-dependent manner) any one of the antibodies disclosed herein, from binding to ApoC3 (e.g., human or cynomolgus ApoC3), is a human antibody. Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., ApoC3 (e.g., human or cynomolgus ApoC3)). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg N & Huszar D (1995) Int Rev Immunol 13:65-93. For a detailed discussion of this technology for producing human antibodies and human monoclonal antibodies and protocols for producing such antibodies, see, e.g., International Publication Nos. WO 98/24893, WO 96/34096 and WO 96/33735; and U.S. Pat. Nos. 5,413,923, 5,625,126, 5,633,425, 5,569,825, 5,661,016, 5,545,806, 5,814,318 and 5,939,598. Examples of mice capable of producing human antibodies include the Xenomouse™ (Abgenix, Inc.; U.S. Pat. Nos. 6,075,181 and 6,150,184), the HuAb-Mouse™ (Mederex, Inc./Gen Pharm; U.S. Pat. Nos. 5,545,806 and 5,569,825), the Trans Chromo Mouse™ (Kirin) and the KM Mouse™ (Medarex/Kirin).

Human antibodies which specifically bind to ApoC3 (e.g., human or cynomolgus ApoC3) can be made by a variety of methods known in the art including phage display methods described above using antibody libraries derived from human immunoglobulin sequences. See also U.S. Pat. Nos. 4,444,887, 4,716,111, and 5,885,793; and International Publication Nos. WO 98/46645, WO 98/50433, WO 98/24893, WO 98/16654, WO 96/34096, WO 96/33735, and WO 91/10741.

In some embodiments, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that specifically bind to a target antigen (e.g., ApoC3 (e.g., human or cynomolgus ApoC3)). Such methods are known and are described in the art, see, e.g., Shinmoto H et al., (2004) Cytotechnology 46: 19-23; Naganawa Y et al., (2005) Human Antibodies 14: 27-31.

### 6. Kits

Also provided, are kits comprising one or more antibodies disclosed herein, or pharmaceutical composition or conjugates thereof. In a specific embodiment, provided herein is a pharmaceutical pack or kit comprising one or more containers filled with one or more of the ingredients of the pharmaceutical compositions disclosed herein, such as one or more antibodies provided herein. In some embodiments, the kits contain a pharmaceutical composition disclosed herein and any prophylactic or therapeutic agent, such as those disclosed herein. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided, are kits that can be used in the above methods. In one embodiment, a kit comprises an antibody disclosed herein, preferably a purified antibody, in one or more containers. In a specific embodiment, kits disclosed herein contain a substantially isolated ApoC3 (e.g., human or cynomolgus ApoC3) antigen as a control. In another specific embodiment, the kits disclosed herein further comprise a control antibody which does not react with an ApoC3 (e.g., human or cynomolgus ApoC3) antigen. In another specific embodiment, kits disclosed herein contain one or more elements for detecting the binding of an antibody to ApoC3 (e.g., human or cynomolgus ApoC3) antigen (e.g., the antibody can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized ApoC3 (e.g., human or cynomolgus ApoC3) antigen. The ApoC3 (e.g., human or cynomolgus ApoC3) antigen provided in the kit can also be attached to a solid support. In a more specific embodiment, the detecting means of the above described kit includes a solid support to which an ApoC3 (e.g., human or cynomolgus ApoC3) antigen is attached. Such a kit can also include a non-attached reporter-labeled anti-human antibody or anti-mouse/rat antibody. In this embodiment, binding of the antibody to the ApoC3 (e.g., human or cynomolgus ApoC3) antigen can be detected by binding of the said reporter-labeled antibody.

### EXAMPLES

Applicants have previously identified a monoclonal antibody that specifically binds to human ApoC3 but does not exhibit appreciable binding to cynomolgus monkey ApoC3 (clone 5E5, set forth in WO/2018/007999). The instant disclosure provides novel anti-ApoC3 antibodies derived from clone 5E5 that specifically bind to both human and cynomolgus ApoC3. The following examples describe the generation and characterization of these novel antibodies. The amino sequences of the novel antibodies are set forth in Tables 1-5, herein.

The examples in this Section are provided to further elucidate the advantages and features of the present application, but are not intended to limit the scope of the application.

### Example 1: Identification of Antibodies that Bind to Both Human and Cynomolgus ApoC3

### 1.1 Generation and Screening of a Light Chain Shuffled Phage Library

The anti-Apoc3 antibody clone, 5E5, was previously isolated from a phage library derived from llamas immunized with human ApoC3 (hereinafter “the 5E5 library”), as described in WO/2018/007999. To identify novel antibodies that specifically bind to both human and cynomolgus ApoC3, a new scFv phage library was generated in which the coding sequences of VL from the 5E5 library were subcloned into the ApaLI and AscI restriction sites of a pSC1 phagemid vector together with the coding sequence of the 5E5 VH, such that individual member of the resultant phage library encoded scFv comprising the 5E5 VH in combination with different VL. The library had a diversity of greater than 10⁸

To enrich for scFv that bound to both human and cynomolgus ApoC3, phage display selections using the light chain shuffled phage display library described above were performed on both native human and/or recombinant cynomolgus ApoC3 (nhuApoC3 and rcyApoC3, respectively). Two separate selection strategies were employed. In a first strategy, the phage display library was selected in a first round of panning selections on nhuApoC3 followed by two consecutive rounds of selection on NeutrAvidin-captured, biotinylated rcyApoC3. In a second strategy, the phage display library was selected in two consecutive rounds on NeutrAvidin-captured, biotinylated rcyApoC3. For all selections, elution was performed with trypsin at neutral pH 7.4. As a control, selections on native huApoC3 were performed in parallel to all selection rounds on rcyApoC3. Phage inputs and outputs were titrated and spotted to determine phage titers and output sizes. Enrichments were calculated and compared to a negative control selection (non-coated well/PBS).

After the phage enrichment described above, individual colonies of phage-infected E. coli TG1 were picked into 96-well master plates (MPs). Periplasmic extracts (containing soluble monoclonal scFv) were prepared from two MPs (MP29 and MP30). The ability of the scFv to biotinylated nhuApoC3 and rcyApoC3 antigens captured at 10 nM on NeutrAvidin-coated wells was determined by ELISA. scFvs were detected using anti-c-myc HRP conjugated antibody (Bethyl #A190-105P) at 1:5000 dilution. A total of 30 clones from MP29 and 76 clones from MP30 were positive for rcyApoC3 binding (OD450 nm values above 0.100 on rcyApoC3).

Positive scFv clones from the ELISA assay described above were then screened using SPR to evaluate their target association and dissociation characteristics. Biotinylated huApoC3 and rcyApoc3 were captured on streptavidin coated chips (SA CHIP; GE Healthcare #BR100398) at pH 7.4 in accordance with the manufacturer's instructions. Briefly, 20 μl of 10 μg/ml biotinylated antigen was injected to reach a surface density of approximately 500 RU. Periplasmic extracts were diluted 1:5 fold in HBS-EP buffer, pH 7.4 (GE Healthcare #BR-1008-26), and 60 μl of each of these diluted extracts was injected and passed through the flow cells at 30 μl/min. An off-rate wash was performed at pH 7.4 for 5 mins. Following dissociation, the flow cell surfaces were regenerated by injecting 10 μl of 10 mM NaOH/1 M NaCl followed by 10 μl of 10 mM glycine at pH 1.5.

The resulting sensorgrams were analyzed with BIAevaluation 4.1 software applying the Langmuir 1:1 binding model to derive off-rate parameters. Data was adjusted to zero and the reference cell sensorgrams were subtracted. Off-rate values were estimated using the dissociation phase of the sensorgrams and compared for each clone for the different antigens. The binding characteristics of selected antibodies are shown in Table 6.

TABLE 6
SPR affinity measurements

| | Binding | | |
|---|---|---|---|
| scFv Clone ID | to | kd (1/s) | R0 |
| 5E5 WT | nhuApoC3 | 9.64E−04 | 854 |
| | rcyApoC3 | 3.91E−02 | 14.1 |
| 29B03 | nhuApoC3 | 5.72E−04 | 930 |
| | rcyApoC3 | 4.18E−02 | 122 |
| 29A06 | nhuApoC3 | 1.62E−03 | 1010 |
| | rcyApoC3 | 4.06E−02 | 125 |
| 29A05 | nhuApoC3 | 6.90E−03 | 545 |
| | rcyApoC3 | 1.46E−02 | 145 |
| 29G02 | nhuApoC3 | 4.35E−03 | 833 |
| | rcyApoC3 | 7.21E−03 | 102 |
| 30D10 | nhuApoC3 | 1.23E−03 | 695 |
| | rcyApoC3 | 4.36E−02 | 11.4 |
| 29G11 | nhuApoC3 | 6.33E−03 | 809 |
| | rcyApoC3 | 7.14E−03 | 92.7 |
| 29F10 | nhuApoC3 | 1.44E−03 | 808 |
| | rcyApoC3 | 5.90E−02 | 52 |

A nhuApoC3-binding clone was considered to also bind rcyApoC3 if the R0 value on rcyApoC3 was above 45 RU (i.e., three fold over the R0 value for WT 5E5 clone). Clones with binding to both human and cynomolgus ApoC3 were sequenced to analyze the diversity of the variable light chains. Sequences of exemplary human and cynomolgus ApoC3-binding antibodies are set forth in Tables 1-5, herein.

### Example 2: IgG Antibody Production and Purification

Based on the data from the ELISA binding assay and the SPR analysis described in Example 1, the scFv clones in Table 6 were selected for IgG formatting and small scale protein production. The scFv clones were reformatted to IgG by cloning each variable region into the BsmBI site of the pCDNA3.1Neo mammalian expression vector.

For antibody productions, 50 ml of ExpiCHO-S cells was transfected with 40 μg of VH and VL pCDNA3.1Neo plasmid DNA and cultured for 8 days. Cells were then removed by centrifugation and supernatant was stored at 4° C.

IgG were purified using HiTrap MabSelect SuRe columns (GE #11-0034-94) on an ÄKTA Pure system. The IgGs were eluted using 0.1 M citrate buffer at pH 3.0 and collected as 1.0 ml fractions in tubes containing 0.1 ml Tris-HCl pH 9.0 for neutralization. Antibody-containing fractions were pooled and desalted in 1×phosphate buffered saline solution (PBS; NaCl 137 mM, KCl 3 mM, Na2HPO4 8 mM, KH2PO4 15 mM, pH7.4) using a HiTrap desalting column on the ÄKTA Pure system. Protein concentration was determined by measuring the absorbance at 280 nm and correcting using the extinction coefficient as follows: (A280 nm-A340 nm)/ε (extinction coefficient in g/L). The size and purity of the samples were confirmed by SDS-PAGE under reducing and non-reducing conditions.

### Example 3: Analysis of Binding Characteristics of Anti-ApoC3 IgG Antibody

In this example, SPR assays of the full length IgG described in Example 2 were performed to evaluate the binding characteristics of the novel light chain antibodies with human and cynomolgus ApoC3. Targets were presented in three formats: immobilized antigen, captured antigen, and captured mAb.

### 3.1 SPR Using Immobilized Antigen

Native huApoC3 protein was immobilized on a CMS chip (GE Healthcare #BR100012). Immobilization was performed in accordance with the methods provided by Biacore using the NHS/EDC kit (Biacore AB). Briefly, following activation of the chip, a solution of 60 μg/ml human ApoC3 in 10 mM acetate buffer at pH 4.5 was injected until the surface density reached approximately 500 RU. Then 60 μL of 1-100 nM test antibody in HBS-EP buffer (GE #BR-1008-26; 0.010 M HEPES, 0.150M NaCl, 3 mM EDTA, 0.05% (v/v) surfactant P20, pH 7.4) was injected and passed through the flow cells at 30 μl/min. An off-rate wash was performed at pH 7.4 for 5 min. Following dissociation, the flow cell surfaces were regenerated by injecting 10 μl of 10 mM NaOH/1 M NaCl and 10 μl of 10 mM glycine at pH 1.5.

The resulting sensorgrams were analyzed with BIAevaluation 4.1 software using the Langmuir 1:1 binding model to derive binding kinetics. Data was zero adjusted and the reference cell sensorgrams were subtracted. An overview of the calculated kinetic parameters for the IgG clones tested is presented in Table 7. Overall, the clones had high affinity for the human target (in the same order as the WT reference 5E5 IgG) and low off-rate (kd<1.0E-4 1/s) values. Specific binding to the cynomolgus target was observed for most of the IgG clones tested, with variable off-rate values.

TABLE 7
SPR results for assays using immobilized antigen

| | | Conc of | ka | kd | | | |
|---|---|---|---|---|---|---|---|
| IgG | Binding | mAh | E+05 | E−04 | Rmax | KD | |
| Clone ID | to | (nM) | (1/Ms) | (1/s) | (RU) | (nM) | Chi2 |
| 5E5 WT | nhuApoC3 | 50-0.8 | n/a | | | | |
| 29B03 | nhuApoC3 | 12.5-0.8 | n/a | | | | |
| | rcyApoC3 | 12.5-0.8 | 3.8 | 0.67 | 460 | 0.18 | 0.7 |
| 29A06 | nhuApoC3 | 12.5-0.8 | 9.5 | 0.65 | 1470 | 0.07 | 49.8 |
| | rcyApoC3 | 12.5-0.8 | 5.0 | 0.75 | 658 | 0.15 | 1.7 |
| 29A05 | nhuApoC3 | 12.5-0.8 | 5.2 | 0.82 | 865 | 0.16 | 2.8 |
| | rcyApoC3 | 12.5-0.8 | 3.1 | 0.64 | 415 | 0.21 | 0.3 |
| 29G02 | nhuApoC3 | 12.5-0.8 | 13.2 | 0.16 | 1290 | 0.01 | 14.9 |
| | rcyApoC3 | 12.5-0.8 | 11.1 | 5.52 | 761 | 0.50 | 21.8 |
| 30D10 | nhuApoC3 | 12.5-0.8 | 15.0 | 2.31 | 1780 | 0.15 | 852.0 |
| | rcyApoC3 | | NS | | | | |
| 29G11 | nhuApoC3 | 12.5-0.8 | 14.4 | 0.63 | 1490 | 0.04 | 131 |
| | rcyApoC3 | 12.5-0.8 | 14.0 | 5.31 | 938 | 0.38 | 50 |
| 29F10 | nhuApoC3 | 50-0.8 | 6.2 | 0.24 | 970 | 0.04 | 292 |
| | rcyApoC3 | 50-0.8 | 2.8 | 7.74 | 467 | 2.79 | 11.1 |

Sensorgrams were blank channel subtracted and double referenced to no-analyte blank assay;
n/a: not applicable;
below the limits of detection kd: 1E−06 1/s;
NS: no binding curve registered

### 3.2 SPR Using Captured Antigen

For the captured antigen approach, biotinylated native human ApoC3 was captured on a streptavidin coated chip (GE Healthcare #BR100032) at pH 7.4. In accordance with the methods provided by Biacore, 20 μl of 10 μg/ml biotinylated human ApoC3 was injected until the surface density reached approximately 500 RU. SPR methods and data analysis were followed as described in Example 2.1. SPR was also performed using these methods with the 29A06-Nhance antibody as the captured antigen. The 29A06-Nhance antibody comprises the heavy chain amino acid sequence set forth in SEQ ID NO: 38 and the light chain amino acid sequence set forth in SEQ ID NO: 50.

An overview of the calculated kinetic parameters for the IgG clones tested is presented in Tables 8 and 9. Similarly to the immobilized antigen approach, most of the clones had high affinity towards the human target and low off-rate values. In addition, specific binding to the cynomolgus target was observed for most of the IgG clones tested, with variable off-rate values.

TABLE 8
SPR results for assays using captured antigen

| | | Conc of | ka | kd | | | |
|---|---|---|---|---|---|---|---|
| IgG | Binding | mAb | E+05 | E−04 | Rmax | KD | |
| Clone ID | to | (nM) | (1/Ms) | (1/s) | (RU) | (nM) | Chi2 |
| 5E5 WT | bio- | 50-0.8 | 3.5 | 0.18 | 475 | 0.05 | 25 |
| | nhuApoC3 | | | | | | |
| 29B03 | bio- | | n/a | | | | |
| | nhuApoC3 | | | | | | |
| | bio- | 12.5-0.8 | 3.2 | 1.04 | 518 | 0.33 | 0.64 |
| | rcyApoC3 | | | | | | |
| 29A06 | bio- | 12.5-0.8 | 9.6 | 0.42 | 1190 | 0.04 | 6.6 |
| | nhuApoC3 | | | | | | |
| | bio- | 12.5-0.8 | 3.7 | 1.69 | 678 | 0.46 | 1.6 |
| | rcyApoC3 | | | | | | |
| 29A05 | bio- | 12.5-0.8 | 5.0 | 0.63 | 762 | 0.13 | 1.4 |
| | nhuApoC3 | | | | | | |
| | bio- | 12.5-0.8 | 2.7 | 1.75 | 475 | 0.65 | 0.57 |
| | rcvApoC3 | | | | | | |
| 29G02 | bio- | 12.5-0.8 | 13.1 | 0.33 | 1100 | 0.02 | 38 |
| | nhuApoC3 | | | | | | |
| | bio- | 12.5-0.8 | 7.5 | 2.75 | 846 | 0.37 | 8.3 |
| | rcyApoC3 | | | | | | |
| 30D10 | bio- | 12.5-0.8 | 12.9 | 1.26 | 1770 | 0.10 | 77 |
| | nhuApoC3 | | | | | | |
| | bio- | | NS | | | | |
| | rcyApoC3 | | | | | | |
| 29G11 | bio- | 12.5-0.8 | 13.8 | 0.73 | 1320 | 0.05 | 37 |
| | nhuApoC3 | | | | | | |
| | bio- | 12.5-0.8 | 9.2 | 2.28 | 1130 | 0.25 | 8.1 |
| | rcyApoC3 | | | | | | |
| 29F10 | bio- | | n/a | | | | |
| | nhuApoC3 | | | | | | |
| | bio- | 50-0.8 | 2.2 | 3.88 | 531 | 1.75 | 31 |
| | rcyApoC3 | | | | | | |

Sensorgrams were blank channel subtracted and double referenced to no-analyte blank assay;
n/a: not applicable;
below the limits of detection kd: 1E−06 1/s;
NS: no binding curve registered

TABLE 9
SPR results for assays using 29A6-Nhance as the captured antigen

| | | KD | | | KD | | | Rmax | Rmax(RU) |
|---|---|---|---|---|---|---|---|---|---|
| | | (pH 7.4, | | | (pH 7.4, | | | (RU) cyno | cyno |
| | Conc of | pM)human | kₐ | k_{d} | pM) cyno | kₐ | k_{d} | apoC-III | apoC3 |
| mAh | analyte | apoC-III | human | human | apoC-III | (cyno) | (cyno) | pH 7.4 | pH 5.5 |
| mAb | 0.78-25 | 23 ± 24 | 7.7E+05 | 4.38E−05 | 675 ± 71 | 2.49E+05 | 1.67E−04 | 1240 | 880 |
| 29A6- | nM | | | | | | | | |
| Nhance | | | | | | | | | |

### 3.3 SPR Using Captured Antibody

Using the inverted assay approach, goat anti-human IgG Fcγ-specific antibody (Jackson ImmunoResearch #109-005-098) was immobilized on a CMS chip (GE Healthcare #BR100012). Immobilization was performed in accordance with the method provided by Biacore in the NHS/EDC kit (Biacore AB). Briefly, after activation of the chip, a solution of 30 μg/ml anti-human Fcγ antibody in 10 mM acetate buffer with pH 5.0 was injected until the surface density reached approximately 10,000 RU. Then 50 nM antibody in HBS-EP buffer (GE #BR-1008-26; 0.010 M HEPES, 0.150M NaCl, 3 mM EDTA, 0.05% (v/v) surfactant P20, pH 7.4) was injected and captured by the high affinity anti-huFcγ antibody at a density up to 800 RUs. Following antibody capture, 100 μl of HBS-EP buffer was injected and passed through the flow cells at 30 μl/min. To bind target, 60 μl of 400 nM, 200 nM, 100 nM, or 50 nM ApoC3 protein in HBS-EP buffer was injected. An off-rate wash was performed by injection of HBS-EP buffer at 30 μl/min for 5 min. After dissociation, the flow cell surfaces were regenerated by injecting 20 μl of 10 mM glycine at pH 1.5.

The resulting sensorgrams were analysed with BIAevaluation 4.1 software using the Langmuir 1:1 binding model to derive binding kinetics. Data was zero adjusted and the reference cell sensorgrams were subtracted. A no-analyte blank assay was used for double referencing the sensorgrams corresponding to nhuApoC3 and rcyApoC3 injections. The dissociation and association phase sensorgrams were fitted separately for the 4 different concentration curves. Sensorgrams were excluded from the fitting if maximum RU values were below the limit of detection (i.e., <5 RU). An overview of the calculated kinetic parameters for the tested IgG clones is presented in Table 10. SPR signals were too low to calculate kinetic parameters for all of the mAbs tested. However, as was observed with the immobilized antigen and captured antigen approaches, most of the clones had high affinity for human ApoC3 and low off-rate values. In addition, specific binding to cynomolgus ApoC3 was observed for several of the light chain IgG clones tested.

TABLE 10
SPR results for assays using captured antibody

| | | Conc of | | | | |
|---|---|---|---|---|---|---|
| IgG | | target | ka | kd | KD | Rmax |
| Clone ID | Target | (nM) | (1/Ms) | (1/s) | (nM) | (RU) |
| 5E5 WT | nhuApoC3 | 50 | 1.64E+05 | 1.14E−04 | 0.695 | 75.2 |
| | | 100 | 1.65E+05 | 1.21E−04 | 0.734 | 75.2 |
| | | 200 | 1.66E+05 | 1.28E−04 | 0.770 | 68.9 |
| | | 400 | 1.65E+05 | 1.22E−04 | 0.741 | 59.5 |
| 29B03 | nhuApoC3 | 50 | | n/a | | |
| | | 100 | 2.38E+05 | 8.00E−05 | 0.336 | 38.7 |
| | | 200 | 2.41E+05 | 8.44E−05 | 0.350 | 36.6 |
| | | 400 | 2.15E+05 | 8.63E−05 | 0.401 | 33.4 |
| | rcyApoC3 | 50 | 6.68E+04 | 0.0127 | 190 | 28 |
| | | 100 | 3.63E+04 | 0.014 | 385 | 52.9 |
| | | 200 | 4.61E+04 | 0.0137 | 297 | 42.3 |
| | | 400 | 3.96E+04 | 0.0145 | 367 | 47.5 |
| 29A06 | nhuApoC3 | 50 | 4.16E+05 | 3.14E−04 | 0.755 | 48.8 |
| | | 100 | 3.90E+05 | 3.17E−04 | 0.814 | 45.6 |
| | | 200 | 3.51E+05 | 3.20E−04 | 0.910 | 42.8 |
| | | 400 | 3.36E+05 | 3.27E−04 | 0.973 | 35.8 |
| | rcyApoC3 | 50 | | | | n/a |
| | | 100 | | | | n/a |
| | | 200 | | | | n/a |
| | | 400 | 2.25E+04 | 0.0207 | 919 | 75.5 |
| 29A05 | nhuApoC3 | 50 | 2.10E+05 | 1.26E−03 | 6.01 | 45.5 |
| | | 100 | 2.06E+05 | 1.32E−03 | 6.42 | 43.5 |
| | | 200 | 1.94E+05 | 1.33E−03 | 6.87 | 42.2 |
| | | 400 | 1.81E+05 | 1.33E−03 | 7.36 | 39.6 |
| | rcyApoC3 | 50 | | | | n/a |
| | | 100 | 3.00E+04 | 8.87E−03 | 296 | 32.3 |
| | | 200 | 2.44E+04 | 8.75E−03 | 359 | 40.1 |
| | | 400 | 2.69E+04 | 9.83E−03 | 365 | 57.1 |
| 29G02 | nhuApoC3 | 50 | 3.11E+05 | 7.18E−04 | 2.31 | 97.8 |
| | | 100 | 3.01E+05 | 7.58E−04 | 2.52 | 102 |
| | | 200 | 2.86E+05 | 7.33E−04 | 2.57 | 103 |
| | | 400 | 2.47E+05 | 7.52E−04 | 3.04 | 107 |
| | rcyApoC3 | 50 | | | | n/a |
| | | 100 | | | | n/a |
| | | 200 | | | | n/a |
| | | 400 | | | | n/a |
| 29G11 | nhuApoC3 | 50 | 2.40E+05 | 1.15E−03 | 4.79 | 106 |
| | | 100 | 2.41E+05 | 1.17E−03 | 4.85 | 107 |
| | | 200 | 2.34E+05 | 1.22E−03 | 5.21 | 109 |
| | | 400 | 2.07E+05 | 1.19E−03 | 5.76 | 117 |
| | rcyApoC3 | 50 | | | | n/a |
| | | 100 | | | | n/a |
| | | 200 | | | | n/a |
| | | 400 | | | | n/a |
| 29F10 | nhuApoC3 | 50 | 1.44E+05 | 2.46E−04 | 1.71 | 83.6 |
| | | 100 | 1.34E+05 | 2.19E−04 | 1.63 | 86.7 |
| | | 200 | 1.33E+05 | 2.17E−04 | 1.63 | 89.2 |
| | | 400 | 1.22E+05 | 2.19E−04 | 1.80 | 93.2 |
| | rcyApoC3 | 50 | | | | n/a |
| | | 100 | | | | n/a |
| | | 200 | | | | n/a |
| | | 400 | | | | n/a |

Sensorgrams were blank channel subtracted and double referenced to no-analyte blank assay;
n/a: not applicable

### Example 4: Pharmacokinetics and Pharmacodynamics of Anti-ApoC3 Antibody 29A06 in an AAV8-huApoC3 Mouse Model

This example describes the in vivo characterization of the anti-ApoC3 antibody 29A06 in a mouse model with impaired triglyceride clearance due to transgenic expression of human ApoC3.

### 4.1 Generation of Mouse Model

Forty 63 day old, male C57Bl6 mice weighing 20-25 g were housed 5 per cage on corn cob bedding with a 12 h day/night cycle. Mice were fed standard rodent chow and allowed water ad libitum. For over expression of human ApoC3, mice were injected intraperitoneally with 250 μL (3.5×1011 genome copies) of a serotype 8 adeno-associated viral vector that expressed human ApoC3 under the liver-specific thyroxine binding globulin (TBG) promoter. The vector was obtained from the Penn Vector Core (Gene Therapy Program of the University of Pennsylvania). Fourteen days following administration, mice were assigned to groups of 6 based on levels of human ApoC3 levels. The mice were bled via the retro-orbital sinus to establish t=0 pre-dose ApoC3 levels. Mice were then immediately given a subcutaneous injection of 30 mg/kg Hyhel5 control antibody, 30 mg/kg pre-germline, pH-dependent 5E5VH5_VL8 antibody (described in PCT/IB2018/052780), or 10, 25, or 50 mg/kg 29A06 test antibody. Mice were bled via the retro-orbital sinus to obtain 25 μL samples of whole blood in 80 μL 0.125% EDTA saline. Samples were used to analyze levels of human ApoC3, mouse ApoC3, triglycerides, and 29A06 antibody at 0 hours, 8 hours, 1 day, and 2 days post-dose.

### 4.2 Pharmacodynamics of Anti-ApoC3 Antibody 29A06

Human ApoC3 ELISA

A 96-well plate (Greiner #655061) was coated with 50 μL of 0.8 μg/mL primary ApoC3 antibody (Abcam rabbit polyclonal anti-human ApoC3 #ab21032) in PBS and incubated overnight at 4° C. The plate was then washed 4 times with 200 μL TBS-T (0.1% Tween-20) and blocked with 200 μL PBS/clear milk/BSA blocking buffer (Pierce Clear Milk Blocker #37587 plus 3% Roche BSA fraction V, protease free #03117332001 or Cell Sciences Native BSA Cohn Fraction V #CSI14635 in PBS) for 90 minutes at 30° C. Blocking buffer was removed and 50 μL of test sample, diluted 1:1200 in blocking buffer, was added. The plate was incubated for 2 hours at room temperature with mixing at 300 rpm. Following incubation, the plate was washed four times with 200 μL TBS-T. Then 50 μL 0.10 μg/mL secondary antibody (Abcam goat polyclonal biotin-conjugate ApoC3 #ab21024) in blocking buffer was added and the plate was incubated for 1 hour at room temperature with mixing at 300 rpm. The plate was washed once with TBS-T, 50 μL SA-HRP (Abcam #34028) diluted 100-fold in PBS was added, and the plate was incubated for 30 minutes at room temperature with mixing at 300 rpm. The plate was then washed 4 times with 200 μL TBS-T, developed with 80 μL TMB (Thermo Ultra-TMB ELISA #34028), and development was terminated with 50 μL 0.5 N HCl. Detection was performed using a spectrophotometer (SpectraMax M5, Molecular Devices) at 450 nm. The amount of ApoC3 in the samples was calculated from a 4-parameter fit of a standard curve (SoftMax Pro Software, Molecular Devices) constructed using purified ApoC3 from human plasma (Athens Research and Technology).

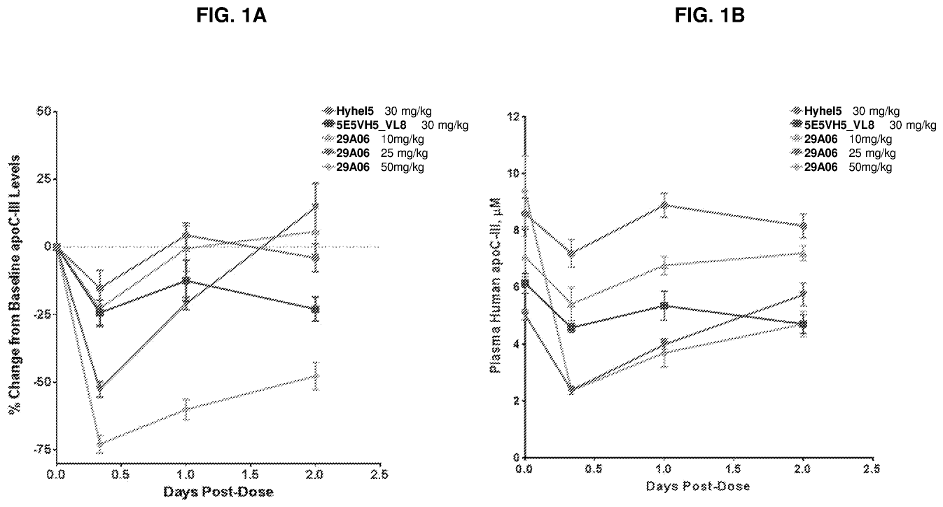

As shown in FIGS. 1A and 1B, administration of the 29A06 antibody produced a dose-dependent reduction of human ApoC3 in mice. For antibody doses of 10, 25 and 50 mg/kg, maximum reductions of approximately 25, 50, and 75%, respectively, were observed within 12 hours. When compared to the pH-dependent 5E5VH5 VL8 antibody, 29A06 had a shorter duration of action. For example, mice treated with 25 mg/kg 29A06 had reduced efficacy by 2 days post-dose, while the 30 mg/kg dose of 5E5VH5 VL8 continued to reduce ApoC3 levels.

### Mouse ApoC3 ELISA

ELISA methods were followed as described above in this Example with the following modifications: the primary ApoC3 antibody (Santa Cruz Biotechnology rabbit polyclonal anti-ApoC3 #SC50378) was used at 0.5 μg/mL, the test sample was diluted 1:100, and the secondary antibody (Abcam goat polyclonal biotin-conjugate ApoC3 #ab21024) was used at 0.6 μg/mL. The amount of mouse ApoC3 in the samples was calculated from a 4-parameter fit of a standard curve (SoftMax Pro Software, Molecular Devices) constructed using recombinant mouse ApoC3 (Blue Sky Bioservices).

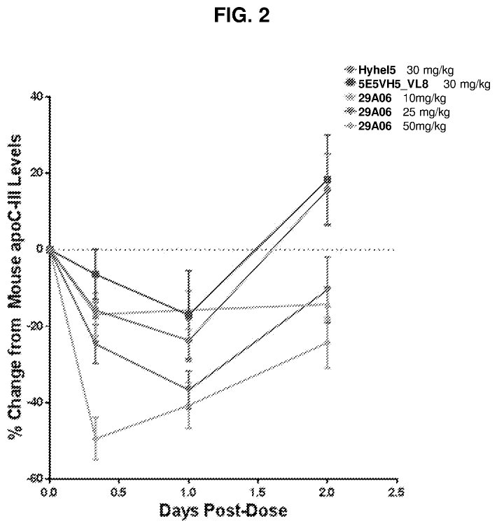

The human and cynomolgus-binding 29A06 antibody produced a dose-dependent reduction of mouse ApoC3 protein, as shown in FIG. 2. Mouse ApoC3 levels decreased by 16, 24, and 49% by 8 hours post-dose for doses of 10, 25 and 50 mg/kg 29A06, respectively. Levels were still reduced by 15, 36 and 40%, respectively, at 24 hours post-dose and remained reduced by 10-24% at 2 days post dose.

### Plasma Triglyceride Assay

Triglycerides were analyzed by incubating 10 μL of diluted plasma with 180 μL Thermo Scientific™ Triglycerides Reagent (#TR22421) in a clear 96 well plate (Corning Costar #9017). After 10 minutes at 37° C., the plate was read at 540 nm on a Spectramax M2 (Molecular Devices) and concentrations were calculated from a linear fit (SoftMax Pro, Molecular Devices) of a glycerol standard curve.

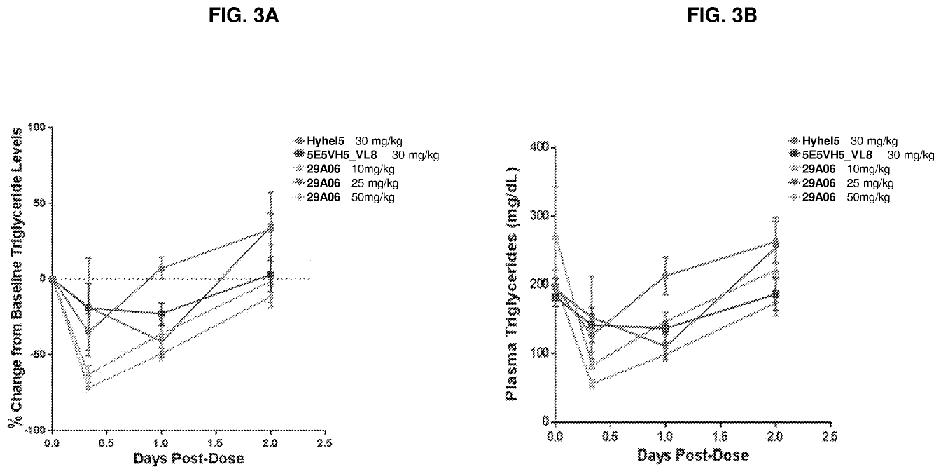

As shown in FIGS. 3A and 3B, plasma triglyceride levels were reduced following 29A06 antibody treatment. At 8 hours post-dose, triglyceride levels were reduced by 63, 18 and 71%, for the doses of 10, 25 and 50 mg/kg of 29A06, respectively. The 71% reduction in triglycerides levels that was observed with the 50 mg/kg dose of 29A06 corresponded to a decrease of approximately 150 mg/dL. Mice treated with 30 mg/kg of the Hyhel5 control antibody experienced an approximately 34% reduction in triglyceride levels at 8 hours post-dose. However, levels returned to baseline by 24 hours post-dose. VH5VL8 decreased triglyceride levels by approximately 22% from 8 to 24 hours post-dose, but levels returned to baseline by 2 days post-dose.

### 4.3 Pharmacokinetics of Anti-ApoC3 Antibody 29A06

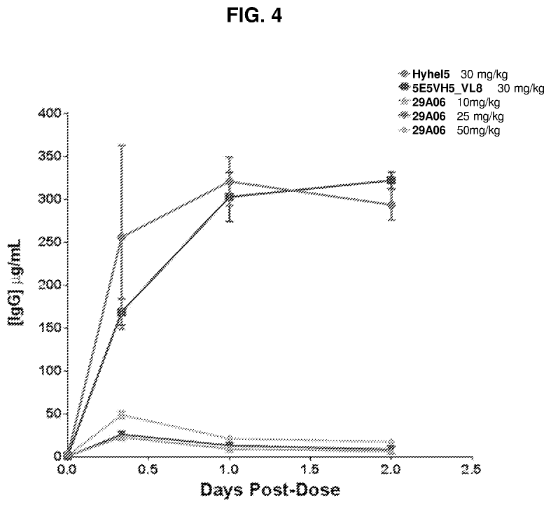

ELISA methods were followed as described above in this Example with the following modifications: the primary IgG antibody (Fitzgerald goat anti-human IgG Fc polyclonal #41-XG57) was used at 2.0 μg/mL, the test sample was diluted 1:1200, and the secondary antibody (Abcam goat anti-human IgG-Fc (biotin) polyclonal #ab97223) was used at 0.05 μg/mL. The amount of IgG in the samples was calculated from a 4-parameter fit of a standard curve (SoftMax Pro, Molecular Devices) constructed using purified test antibody. As shown in FIG. 4, plasma IgG levels increased and peaked around 8 hours post-dosing and then decreased.

### Example 5: Pharmacokinetics and Pharmacodynamics of Anti-ApoC3 Antibody 29A06-Nhance in a Cynomolgus Model

This example describes the in vivo characterization of the anti-ApoC3 antibody 29A06-Nhance in cynomolgus monkey model. The 29A06-Nhance antibody comprises the heavy chain amino acid sequence set forth in SEQ ID NO: 38 and the light chain amino acid sequence set forth in SEQ ID NO: 50.

Five 3-4 kg, non-naïve male cynomolgus monkeys (Macaca fascicularis) were included in this study and may have been previously dosed with small molecules. Two animals were used only to determine the levels of circulating ApoC3 prior to treatment. Animals stayed with their original cage mate or group, housed (up to 3/sex/group/cage) in stainless steel cages, with an automatic watering system. Housing conditions were maintained unless deemed inappropriate by the Study Director and/or Site Veterinarian at Charles River. Animals were separated during designated procedures. Following study completion, all animals were returned to the Testing Facility colony for future use following a suitable washout and recovery period. Animals were fed PMI Nutrition International Certified Primate Chow No. 5048 twice daily. Supplemental diet was provided to the animals as warranted by clinical signs or other changes. At the time of study, animals were fasted overnight with food removal by 7 PM the prior day. Fasting was maintained for 10-12 hours before all blood collections, including the pre-study collection, and then morning food was retuned. Evening food was provided per Testing Facility standard operating procedures. Body weights were taken and recorded before pre-study blood collection and prior to dosing with 29A06-Nhance antibody on Day 1, Day 8 and Day 22. Dosing was administered as shown in Table 11.

TABLE 11
Cynomolgus dosing with 29A06-Nhance antibody

| | Pre-study | | | | | | Dose | | | |
|---|---|---|---|---|---|---|---|---|---|---|
| | blood | Dose | No of | Test | Dose | Concentration | Volume | | | |
| Group | collection\hat{ } | Date | Males | Compound | (mg/kg) | (mg/mL) | (mL/kg) | Route | Vehicle | Flush |
| 1 | Day −5 | Day | 3 | 29A06- | 50 | 40 | 1.25 | IV | PBS | 3 mL |
| | to −10 | 1, 8 | | NHANCE | | | | | | Saline |
| | | and | | | | | | | | |
| | | 22 | | | | | | | | |
| 3* | Day −5 | NA | 2* | NA | NA | NA | NA | NA | NA | NA |
| | to −10 | | | | | | | | | |

*for pre-study blood collection only,
\hat{ }one collection only.

All blood samples were collected from a peripheral vein that was not used for intravenous dosing. Approximately 1 mL of whole blood was collected at each time point: Pre-Study, 0, 24, 48, 72, 96, 120, 144, and 168 hours post-dose. Whole blood samples were stored at room temperature for at least 30 minutes and no longer than 60 minutes. Serum was frozen within 75 minutes of collection and stored at −80° C. until analysis.

### 5.1 Pharmacodynamics of Anti-ApoC3 Antibody 29A06-Nhance

### Cynomolgus ApoC3 ELISA

ELISA methods were followed as described in Example 4 with the following modifications: the primary ApoC3 antibody (in-house anti-human, cynomolgus-binding; Class 14C07) was used at 1.5 μg/mL, the test sample was diluted 1:15000, and the secondary antibody (Abcam goat polyclonal biotin-conjugate ApoC3 #ab21024) was used at 0.25 μg/mL. The amount of ApoC3 in the samples was calculated from a 4-parameter fit of a standard curve (SoftMax Pro, Molecular Devices) constructed using recombinant cynomolgus ApoC3 (Blue Sky Bioservices).

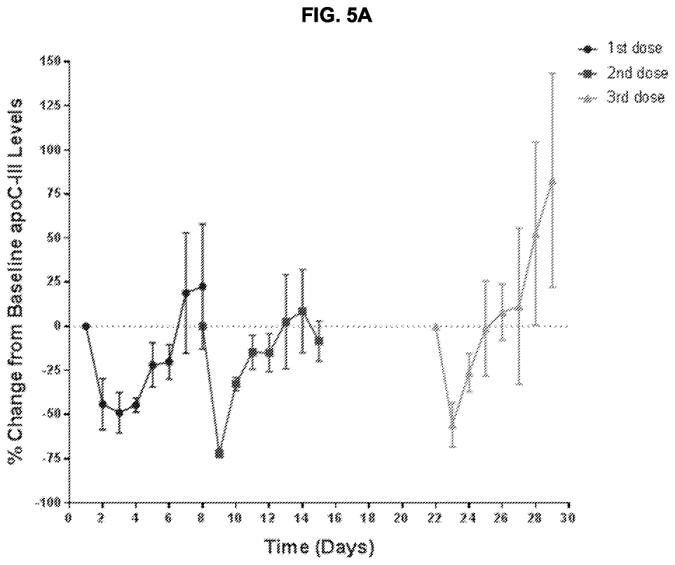

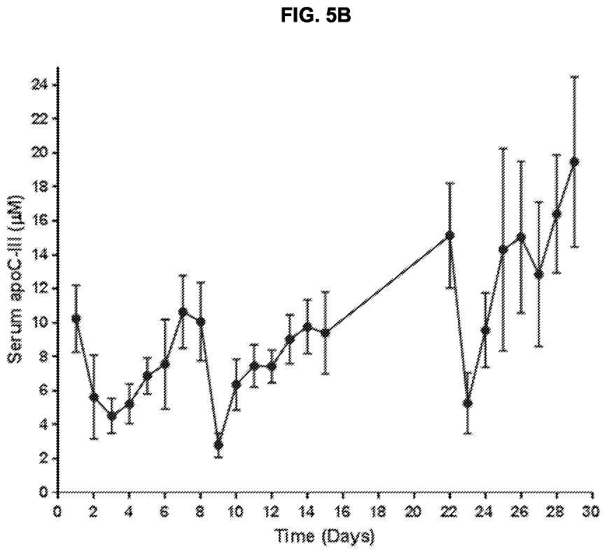

Serum levels of ApoC3 were reduced following administration of 29A06-Nhance, as shown in FIGS. 5A and 5B. A 44% reduction was observed 24 hours after the first dose. However, ApoC3 levels returned to pre-dose levels between days 5 and 6 post-dose. Following administration of the second dose on day 8, serum levels of ApoC3 were reduced by 72% at 24 hours post-dose. Again, ApoC3 levels returned to baseline 5 days post-dose. Similarly, after the third dose on day 22, serum ApoC3 levels were reduced by 55% 24 hours post-dose with levels returning to baseline between days 3 and 4 post-dose.

### Cynomolgus ApoB ELISA

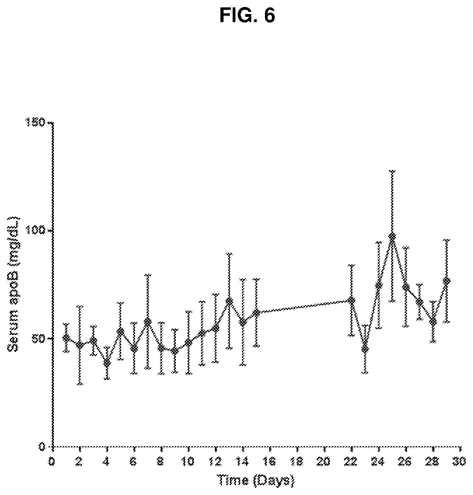

ELISA methods were followed as described in Example 4 with the following modifications: the primary ApoB antibody (Meridian Life Sciences goat polyclonal anti-human ApoB #K45253G) was used at 2.0 μg/mL, test sample was diluted 1:2000, and the secondary antibody (Meridian Life Sciences goat polyclonal biotin-conjugate ApoB 48/100 #34003G) was used at 0.75 μg/mL. The amount of mouse ApoB in the samples was calculated from a 4-parameter fit of a standard curve (SoftMax Pro Software, Molecular Devices) constructed using mouse VLDL (prepared in-house using the OptiPrep methodology), assuming that ApoB is 20% of total VLDL weight. Serum ApoB levels were unchanged from their baseline, pre-dose values, as shown in FIG. 6.

### Plasma Triglyceride Assay

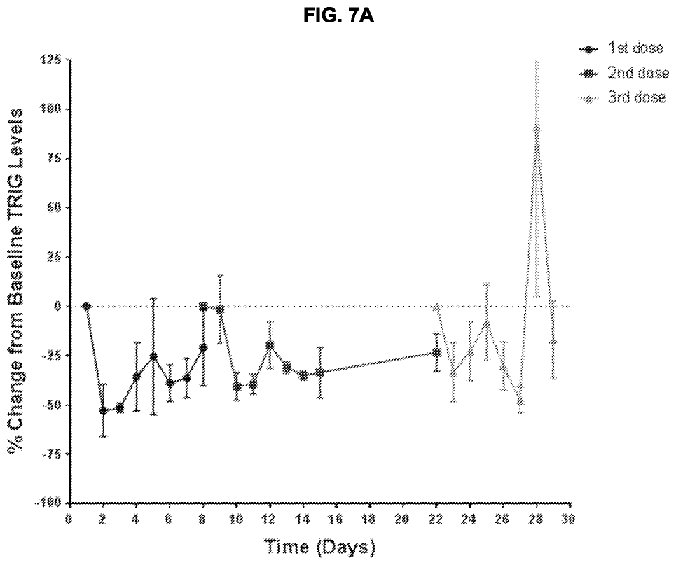

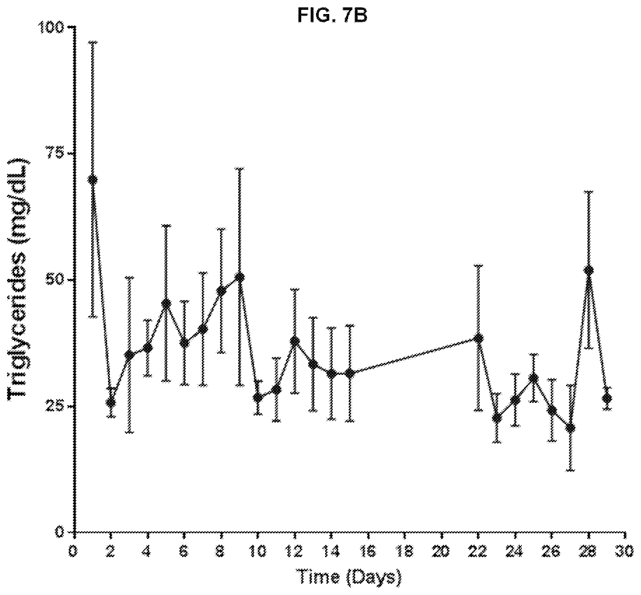

Methods were followed as described in Example 4. As shown in FIGS. 7A and 7B, serum triglyceride levels decreased by 52, 40 and 33% for each of the three doses, respectively, 24-48 hours following administration. However, these changes were not significant, likely due to the variable pre-dose levels in this limited number of animals.

### 5.2 Pharmacokinetics of Anti-ApoC3 Antibody 29A06-Nhance

ELISA methods were followed as described in Example 3 with the following modifications: the primary IgG antibody (Abcam Ab99771, mouse monoclonal 4E3 anti-human IgG1 hinge heavy chain) was used at 1.5 μg/mL, the test sample was diluted 1:9000, and the secondary antibody (Abcam Goat F(ab′)2 Anti-Human IgG-Fc (HRP), pre-adsorbed (ab98595)) was used at 0.1 μg/mL. The amount of IgG in the samples was calculated from a 4-parameter fit of a standard curve (SoftMax Pro, Molecular Devices) constructed using purified test antibody.

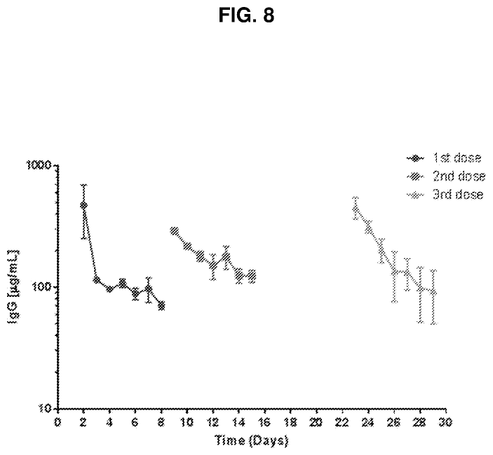

Serum levels of 29A06-Nhance IgG were 473 μg/mL at 24 hours after the first dose. The levels remained around 100 μg/mL until 6 days post-dose then dropped to around 71 μg/mL, as shown in FIG. 8. Following dose 2 on day 8, serum levels were 291 μg/mL at 24 hours post-dose. Levels stayed above 100 μg/mL for 7 days post-dose. Similarly, 24 hours after dose 3 on day 22, serum IgG levels were 456 μg/mL and levels were maintained around 100 μg/mL.

1. An isolated antibody that specifically binds to human and cynomologus monkey ApoC3, wherein the antibody specifically binds to an epitope within the amino acid sequence FSEFWDLDP (SEQ ID NO: 3).

2. The isolated antibody of claim 1, wherein the antibody specifically binds to:
(a) an epitope within the amino acid sequence LSGFWDLNP (SEQ ID NO: 4)
(b) at least one of the amino acids at position 2, 5, or 6 of SEQ ID NO: 3;
(c) positions 2 and 5 of SEQ ID NO: 3;
(d) positions 2 and 6 of SEQ ID NO: 3;
(e) positions 5 and 6 of SEQ ID NO: 3; or
(f) positions 2, 5, and 6 of SEQ ID NO: 3.

3-4. (canceled)

5. An isolated antibody that specifically binds to human and cynomologus monkey ApoC3, comprising a heavy chain variable region comprising complementarity determining regions CDRH1, CDRH2 and CDRH3, and a light chain variable region comprising complementarity determining regions CDRL1, CDRL2 and CDRL3, wherein:
(a) CDRH1 comprises the amino acid sequence TYSMR (SEQ ID NO: 5);
(b) CDRH2 comprises the amino acid sequence SISTDGGGTAYRDSVKG (SEQ ID NO: 6);
(c) CDRH3 comprises the amino acid sequence AGYSD (SEQ ID NO: 7);
(d) CDRL1 comprises the amino acid sequence X₁AX₂QX₃LX₄X₅X₆X₇GX₈TYLY (SEQ ID NO: 22), wherein
X₁ is K or T,
X₂ is G, S or T,
X₃ is N or S,
X₄ is V or R,
X₅ is H or Y,
X₆ is I, P or S,
X₇ is D or N, and
X₈ is K or R;
(e) CDRL2 comprises the amino acid sequence X₁VSX₂RX₃S (SEQ ID NO: 23), wherein
X₁ is D or G,
X₂ is N or T, and
X₃ is D, G or P; and
(f) CDRL3 comprises the amino acid sequence AQX₁TYX₂X₃X₄T (SEQ ID NO: 24), wherein
X₁ is D or G,
X₂ is S, W or Y,
X₃ is P or T, and
X₄ is K or L.

6. The isolated antibody of claim 5, wherein:
(a) CDRL1 comprises an amino acid sequence selected from the group consisting of SEQ ID NO: 8, 9, 10, 11, 12 and 13;
(b) CDRL2 comprises an amino acid sequence selected from the group consisting of SEQ ID NO: 14, 15, 16, 17, and 18; and
(c) CDRL3 comprises an amino acid sequence selected from the group consisting of SEQ ID NO: 19, 20, and 21.

7. The isolated antibody of claim 5, wherein the antibody comprises a heavy chain variable region comprising complementarity determining regions CDRH1, CDRH2 and CDRH3, and a light chain variable region comprising complementarity determining regions CDRL1, CDRL2 and CDRL3, wherein CDRH1, CDRH2, CDRH3, CDRL1, CDRL2, and CDRL3 comprise the amino acid sequences set forth in SEQ ID NOs: 5, 6, 7, 8, 14, and 19; 5, 6, 7, 9, 15, and 19; 5, 6, 7, 10, 14, and 19; 5, 6, 7, 11, 16, and 20; 5, 6, 7, 12, 17, and 21; 5, 6, 7, 13, 15, and 19; or 5, 6, 7, 10, 18, and 20, respectively.

8. The isolated antibody of claim 5, wherein the antibody comprises a light chain variable region comprising an amino acid sequence selected from the group consisting of SEQ ID NOs: 27-33 or a heavy chain variable region comprising the amino acid sequence set forth in SEQ ID NO: 25.

9. An isolated antibody that specifically binds to ApoC3, the antibody comprising a light chain variable region comprising an amino acid sequence selected from the group consisting of SEQ ID NOs: 27-33.

10. The isolated antibody of claim 5, wherein the antibody comprises a heavy chain variable region and a light chain variable region, wherein the heavy chain variable region and the light chain variable region, respectively, comprise the amino acid sequences set forth in SEQ ID NOs: 25 and 27, 25 and 28, 25 and 29, 25 and 30, 25 and 31, 25 and 32, or 25 and 33.

11. The isolated antibody of claim 1, wherein the antibody comprises
(a) a human lambda or human kappa light chain constant region; and/or
(b) a heavy chain constant region, optionally a human IgG1, IgG2, or IgG4 constant region, optionally wherein
(i) the heavy chain constant region is a variant of a wild type human immunoglobulin heavy chain constant region, and wherein the variant human immunoglobulin heavy chain constant region has an increased affinity for human neonatal Fc receptor (FcRn) at pH 6 relative to the affinity of the corresponding wild type human immunoglobulin heavy chain constant region for human FcRn at pH 6,
(ii) the heavy chain constant region comprises the amino acids K, F, and Y at EU positions 433, 434, and 436, respectively,
(iii) the heavy chain constant region comprises the amino acids Y, T, and E at EU positions 252, 254, and 256, respectively,
(iv) the heavy chain constant region comprises the amino acids L and S at EU positions 428 and 434, respectively, or
(v) the heavy chain constant region is an IgG4 constant region comprising the amino acid P at EU position 228.

12. The isolated antibody of claim 11, wherein the antibody comprises:
(a) a light chain comprising the amino acid sequence set forth in SEQ ID NO: 50, 51, 52, 53, 54, 55, or 56; and/or
(b) a heavy chain comprising of the amino acid sequence set forth in SEQ ID NO: 34, 35, 36, 37, 38, 39, 40, 41, 42, 43, 44, 45, 46, 47, 48, or 49.

13-19. (canceled)

20. The isolated antibody of claim 5, wherein the antibody comprises a heavy chain and a light chain, wherein the amino acid sequences of the heavy chain and the light chain, respectively, comprise or consist of the amino acid sequences set forth in SEQ ID NOs: 34 and 50, 35 and 50, 36 and 50, 37 and 50, 38 and 50, 39 and 50, 40 and 50, 41 and 50, 42 and 50, 43 and 50, 44 and 50, 45 and 50, 46 and 50, 47 and 50, 48 and 50, or 49 and 50.

21. The isolated antibody of claim 1, wherein:
(a) the antibody is capable of binding to lipid-bound ApoC3;
(b) the antibody attenuates the ability of ApoC3 to inhibit hepatocyte uptake of very low density lipoprotein (VLDL);
(c) the antibody is capable of increasing the rate of clearance of ApoC3 from the blood in a subject;
(d) the antibody is capable of reducing the level of ApoC3 in the blood in a subject; and/or
(e) the antibody is capable of inhibiting post-prandial lipemia in a subject.

22-25. (canceled)

26. A pharmaceutical composition comprising the antibody of claim 1 and a pharmaceutically acceptable carrier.

27. A polynucleotide encoding the heavy chain variable region and/or the light chain variable region of the antibody of claim 1.

28. An expression vector comprising the polynucleotide of claim 27.

29. A host cell comprising the expression vector of claim 28.

30. A method for producing an antibody that binds to ApoC3, the method comprising culturing the host cell of claim 29 under conditions that allow expression of the antibody.

31. A method for:
(a) inhibiting the activity of ApoC3 in the blood of a subject;
(b) reducing triglyceride levels in the blood of a subject;
(c) inhibiting post-prandial lipemia in a subject; and/or
(d) treating hypertriglyceridemia in a subject,
wherein the method comprising administering to the subject an effective amount of the antibody of claim 1.

32-34. (canceled)

35. A method for treating chylomicronemia in a subject, the method comprising administering to the subject an effective amount of the antibody of claim 1, optionally wherein:
(a) the antibody reduces the levels of chylomicron or chylomicron remnants in the blood of the subject; and/or
(b) the subject is receiving an additional lipid lowering agent, optionally wherein
(i) the additional lipid lowering agent is an HMG-CoA reductase inhibitor, optionally wherein the HMG-CoA reductase inhibitor is atorvastatin, fluvastatin, lovastatin, pitavastatin, pravastatin, rosuvastatin or simvastatin,
(ii) the additional lipid lowering agent is a PCSK9 inhibitor, optionally wherein the PCSK9 inhibitor is alirocumab, evolocumab, or bococizumab,
(iii) the additional lipid lowering agent is ezetimibe,
(iv) the additional lipid lowering agent is a combination of ezetimibe and an HMG-CoA reductase inhibitor, and/or
(v) the additional lipid lowering agent is a combination of ezetimibe, an HMG-CoA reductase inhibitor, and a PCSK9 inhibitor.

36. A method for reducing the risk of cardiovascular disease in a subject with hypertriglyceridemia, the method comprising administering to the subject an effective amount of the antibody of claim 1, optionally wherein:
(a) the cardiovascular disease is myocardial infarction;
(b) the cardiovascular disease is angina;
(c) the cardiovascular disease is stroke; and/or
(d) the cardiovascular disease is atherosclerosis.

37-49. (canceled)
